(19) United States
(12) Patent Application Publication
(10) Pub. No.: US 2012/0172598 A1
(43) Pub. Date: Jul. 5, 2012

(54) COMPOUNDS HAVING TAFIa INHIBITORY ACTIVITY

(75) Inventors: Hideaki Amada, Toshima-ku (JP); Daisuke Matsuda, Toshima-ku (JP); Ayako Bohno, Toshima-ku (JP)

(73) Assignee: TAISHO PHARMACEUTICAL CO., LTD., Toshima-ku, Tokyo (JP)

(21) Appl. No.: 13/496,625

(22) PCT Filed: Sep. 17, 2010
(86) PCT No.: PCT/JP2010/066637

§ 371 Date: Mar. 16, 2012

(30) Foreign Application Priority Data
Sep. 17, 2009 (JP) 2009-215093
Apr. 24, 2010 (JP) 2010-103546

(51) Int. Cl. C07D 471/04

(52) U.S. Cl. 546/84

(57) ABSTRACT

The present invention provides compounds having superior TAFIa inhibitory activity. They are dihydroimidazoquinoline compounds represented by the following formula (I) or pharmaceutically acceptable salts thereof:

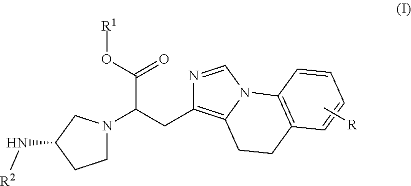

wherein R is a hydrogen atom or a C₁₋₁₀ alkyl group; R¹ is a hydrogen atom, a C₁₋₁₀ alkyl group, a C₃₋₈ cycloalkyl group or a substituent having the structure represented by the following formula Ia or Ib:

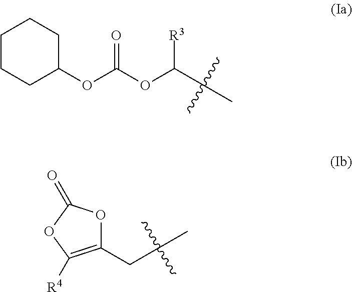

where R³ is a C₁₋₆ alkyl group; R⁴ is a C₁₋₆ alkyl group, a C₃₋₈ cycloalkyl group, or a benzyl group; and R² is a hydrogen atom or a substituent having the structure represented by the following formula Ic or Id:

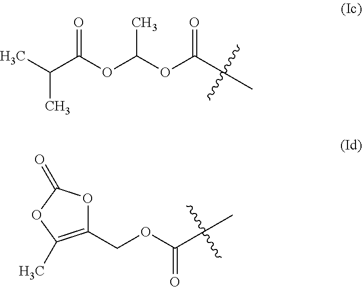

### TECHNICAL FIELD

The present invention relates to compounds having TAFIa (thrombin-activated thrombin-activatable fibrinolysis inhibitor) inhibitory activity.

### BACKGROUND ART

Thrombin-activatable fibrinolysis inhibitor (TAFI) is a carboxypeptidase that is activated by thrombin and thrombomodulin to cleave the lysine residues at the C terminus of the α-chain of fibrin. On the fibrin clot, tissue plasminogen activator (t-PA) and plasminogen bind to the lysine residues at the C terminus of the α-chain of fibrin, whereby plasmin is generated efficiently and fibrinolysis is eventually promoted. On the other hand, TAFIa decreases the affinity of t-PA and plasminogen for the fibrin clot and fibrinolysis activity through the cleavage of lysine residue at the C terminus of the fibrin clot. Hence, TAFIa inhibitors, which efficiently enhance the dissolution of fibrin clots but do not directly inhibit coagulation factors, are expected to contribute to the discovery of antithrombotics or fibrinolysis promoters that have higher clot specificity than the conventional anticoagulants and thrombolytics. Thereby, TAFIa inhibitors are expected to be anti-thrombosis agents that present a lower risk for bleeding and feature higher safety.

Several compounds have heretofore been reported as TAFIa inhibitors and they include thiol derivatives, phosphoric acid derivatives, imidazole derivatives and urea derivatives, all chelating with zinc at the active center of the enzyme (see PTL 1-14 and NPL 1-8). However, nothing has been known about tricyclic compounds typified by dihydroimidazoquinoline derivatives which are related to the compounds of the present invention. In addition, those known TAFIa inhibitors are not considered to have adequate activity and it is desired to develop compounds that have therapeutic effects based on the TAFIa inhibitory action and which hence are satisfactory as pharmaceuticals.

### CITATION LIST

### Patent Literature

PTL 1: Pamphlet of International Publication WO2000/066557
PTL 2: Pamphlet of International Publication WO2000/066550
PTL 3: Pamphlet of International Publication WO2001/019836
PTL 4: Pamphlet of International Publication WO2002/014285
PTL 5: Pamphlet of International Publication WO2003/106420
PTL 6: Pamphlet of International Publication WO2003/027128
PTL 7: Pamphlet of International Publication WO2003/013526
PTL 8: Pamphlet of International Publication WO2003/061652
PTL 9: Pamphlet of International Publication WO2003/061653
PTL 10: Pamphlet of International Publication WO2003/080631
PTL 11: Pamphlet of International Publication WO2005/105781
PTL 12: Pamphlet of International Publication WO2007/045339
PTL 13: Pamphlet of International Publication WO2008/067909
PTL 14: Pamphlet of International Publication WO2009/146802

### Non Patent Literature

NPL 1: J. Med. Chem., Vol. 46, No. 25, pp. 5294-5297, 2003
NPL 2: Bioorganic & Medicinal Chemistry, Vol. 12, No. 5, pp. 1151-1175, 2004
NPL 3: Bioorganic & Medicinal Chemistry Letters, Vol. 14, No. 9, pp. 2141-2145, 2004
NPL 4: J. Pharmacol., Exp., Ther., Vol. 309, No. 2, pp. 607-615, 2004
NPL 5: J. Med. Chem., Vol. 50, No. 24, pp. 6095-6103, 2007
NPL 6: Bioorganic & Medicinal Chemistry Letters, Vol. 17, No. 5, pp. 1349-1354, 2007
NPL 7: Current Opinion in Drug & Development, Vol. 11, No. 4, pp. 480-486, 2008
NPL 8: Bioorganic & Medicinal Chemistry Letters, Vol. 20, No. 1, pp. 92-96, 2010

### SUMMARY OF INVENTION

### Technical Problem

An object of the present invention is to provide compounds having superior TAFIa inhibitory activity.

### Solution to Problem

The present inventors conducted intensive studies with a view to attaining the stated object and found that compounds represented by the following formula (I) have superior TAFIa inhibitory activity. Some of the compounds represented by formula (I) are prodrugs for other compounds of formula (I). In the section of EXAMPLES, a 2-[(3S)-3-aminopyrrolidin-1-yl]-3-(4,5-dihydroimidazo[1,5-a]quinolin-3-yl)propanoic acid derivative was chosen as an exemplary prodrug and subjected to an animal experiment, whereupon this type of prodrug was found to increase the in vivo exposure level of the parent compound. The present invention has been accomplished on the basis of this finding.

Briefly, the present invention provides a dihydroimidazoquinoline compound represented by the following formula (I) or a pharmaceutically acceptable salt thereof:

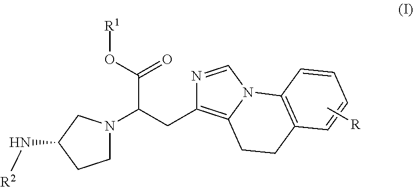

wherein R is a hydrogen atom or a C₁₋₁₀ alkyl group; R¹ is a hydrogen atom, a C₁₋₁₀ alkyl group, a C₃₋₈ cycloalkyl group or a substituent having the structure represented by the following formula Ia or Ib:

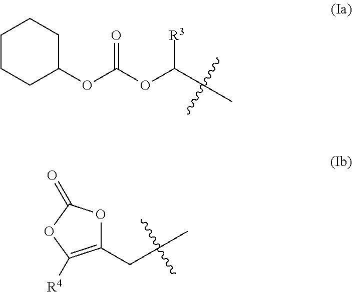

where R³ is a C₁₋₆ alkyl group;
R⁴ is a C₁₋₆ alkyl group, a C₃₋₈ cycloalkyl group, or a benzyl group; and
R² is a hydrogen atom or a substituent having the structure represented by the following formula Ic or Id:

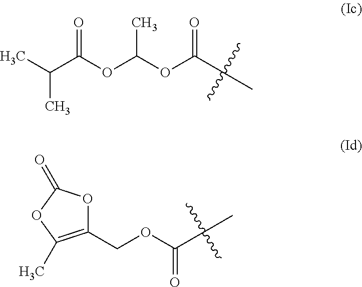

In another embodiment of the present invention, the dihydroimidazoquinoline compound of formula (I) or a salt thereof is a dihydroimidazoquinoline compound represented by the following formula (II) or a pharmaceutically acceptable salt thereof:

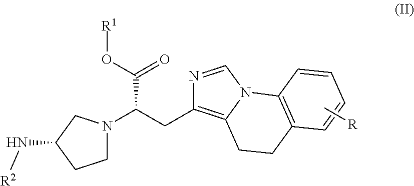

wherein R, R¹ and R² are as defined above in connection with formula (I).

The steric configuration of the asymmetric carbon atom at 2-position in the 3-(4,5-dihydroimidazo[1,5-a]quinolin-3-yl)propanoic acid in formula (II) is the (S)-configuration.

In another embodiment of the present invention, the dihydroimidazoquinoline compound of formula (II) or a salt thereof is a dihydroimidazoquinoline compound represented by the following formula (III) or a pharmaceutically acceptable salt thereof:

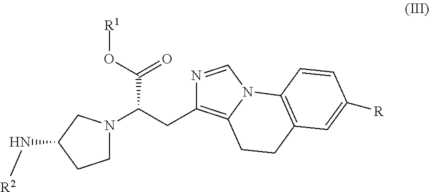

wherein R, R¹ and R² are as defined above in connection with formula (II).

The steric configuration of the asymmetric carbon atom at 2-position in the 3-(4,5-dihydroimidazo[1,5-a]quinolin-3-yl)propanoic acid in formula (III) is the (S)-configuration.

In addition, the position of substitution of R on the dihydroimidazoquinoline ring in formula (III) is 7-position.

In another embodiment of the present invention, the dihydroimidazoquinoline compound of formula (III) or a salt thereof is a dihydroimidazoquinoline compound represented by the following formula (IV) or a pharmaceutically acceptable salt thereof:

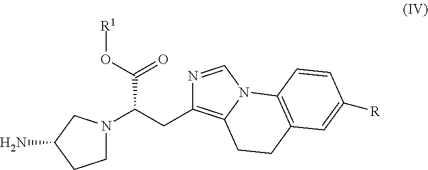

wherein R and R¹ are as defined above in connection with formula (III).

The steric configuration of the asymmetric carbon atom at 2-position in the 3-(4,5-dihydroimidazo[1,5-a]quinolin-3-yl)propanoic acid in formula (IV) is the (S)-configuration.

In addition, the position of substitution of R on the dihydroimidazoquinoline ring in formula (IV) is 7-position.

In another embodiment of the present invention, the dihydroimidazoquinoline compound of formula (III) or a salt thereof is a dihydroimidazoquinoline compound represented by the following formula (V) or a pharmaceutically acceptable salt thereof:

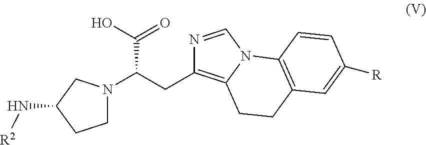

wherein R and R² are as defined above in connection with formula (III).

The steric configuration of the asymmetric carbon atom at 2-position in the 3-(4,5-dihydroimidazo[1,5-a]quinolin-3-yl)propanoic acid in formula (V) is the (S)-configuration.

In addition, the position of substitution of R on the dihydroimidazoquinoline ring in formula (V) is 7-position.

In another embodiment of the present invention, the dihydroimidazoquinoline compound of formula (I) or a salt thereof is a compound represented by the following formula (VI) or a pharmaceutically acceptable salt thereof:

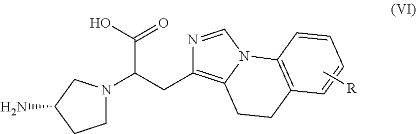

wherein R is as defined above in connection with formula (I).

In another embodiment of the present invention, the dihydroimidazoquinoline compound of formula (VI) or a salt thereof is a dihydroimidazoquinoline compound represented by the following formula (VII) or a pharmaceutically acceptable salt thereof:

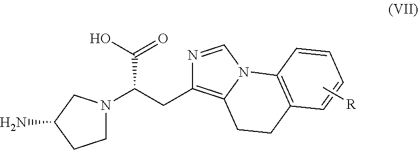

wherein R is as defined above in connection with formula (VI).

The steric configuration of the asymmetric carbon atom at 2-position in the 3-(4,5-dihydroimidazo[1,5-a]quinolin-3-yl)propanoic acid in formula (VII) is the (S)-configuration.

In another embodiment of the present invention, the dihydroimidazoquinoline compound of formula (VII) or a salt thereof is a dihydroimidazoquinoline compound represented by the following formula (VIII) or a pharmaceutically acceptable salt thereof:

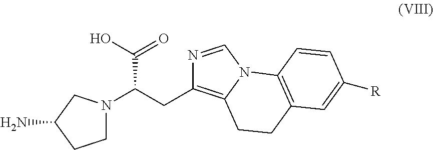

wherein R is as defined above in connection with formula (VII).

The steric configuration of the asymmetric carbon atom at 2-position in the 3-(4,5-dihydroimidazo[1,5-a]quinolin-3-yl)propanoic acid in formula (VIII) is the (S)-configuration.

In addition, the position of substitution of R on the dihydroimidazoquinoline ring in formula (VIII) is 7-position.

### Advantageous Effects of Invention

According to the present invention, compounds having superior TAFIa inhibitory activity can be provided.

### DESCRIPTION OF EMBODIMENTS

The present invention provides compounds of formulas (I) to (VIII) having superior TAFIa inhibitory activity or pharmaceutically acceptable salts thereof.

In formulas (I), (II), (VI) and (VII), the position of substitution of R is not limited but it is preferably located at 7-position on the dihydroimidazoquinoline ring.

In formula (IV), R is preferably a hydrogen atom or a methyl group; R¹ is preferably a hydrogen atom or a substituent having the structure represented by the following formula Ib (where R⁴ is a C₁₋₆ alkyl group, a C₃₋₆ cycloalkyl group, or a benzyl group), more preferably a hydrogen atom or a substituent having the structure represented by the following formula Ib (where R⁴ is an isobutyl group, a tert-butyl group, a cyclohexyl group, or a benzyl group).

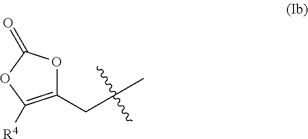

On the following pages, the compounds of the present invention are described in greater detail.

The “C₁₋₆ alkyl group” refers to linear or branched alkyl groups having 1 to 6 carbon atoms. Examples include a methyl group, an ethyl group, a n-propyl group, an isopropyl group, a n-butyl group, an isobutyl group, a sec-butyl group, a tert-butyl group, a n-pentyl group, an isopentyl group, a neopentyl group, a n-hexyl group, and an isohexyl group.

The “C₁₋₁₀ alkyl group” refers to linear or branched alkyl groups having 1 to 10 carbon atoms. Examples include a methyl group, an ethyl group, a n-propyl group, an isopropyl group, a n-butyl group, an isobutyl group, a sec-butyl group, a tert-butyl group, a n-pentyl group, an isopentyl group, a neopentyl group, a n-hexyl group, an isohexyl group, a n-heptyl group, a n-octyl group, a n-nonyl group, and a n-decyl group.

The “C₃₋₈ cycloalkyl group” refers to cyclic alkyl groups having 3 to 8 carbon atoms. Examples include a cyclopropyl group, a cyclobutyl group, a cyclopentyl group, a cyclohexyl group, a cycloheptyl group, and a cyclooctyl group.

The compounds of the present invention are tricyclic compounds having the dihydroimidazoquinoline ring or they may be pharmaceutically acceptable salts of such compounds (either type is hereinafter called “the compounds of the present invention” as appropriate).

Examples of the pharmaceutically acceptable salts include acid addition salts such as mineral acid salts (e.g. hydrochloride, hydrobromide, hydroiodide, phosphate, sulfate, and nitrate), sulfonic acid salts (e.g. methanesulfonate, ethanesulfonate, benzenesulfonate, p-toluenesulfonate, and trifluoromethanesulfonate), and organic acid salts (e.g. oxalate, tartrate, citrate, maleate, succinate, acetate, benzoate, mandelate, ascorbate, lactate, gluconate, and malate); amino acid salts such as glycine salt, lysine salt, arginine salt, ornithine salt, glutamic acid salt, and aspartic acid salt; and inorganic salts such as lithium salt, sodium salt, potassium salt, calcium salt and magnesium salt, as well as salts with organic bases, as exemplified by ammonium salt, triethylamine salt, diisopropylamine salt, and cyclohexylamine salt. The salts may be hydrate salts.

Some of the compounds of the present invention are prodrugs. Specifically, those compounds of formula (I) or (II) in which either R¹ or R² or both are other than a hydrogen atom undergo enzymatic or chemical hydrolysis in vivo so that the amino group and the carboxyl group are deprotected, yielding compounds in which R¹ and R² are both a hydrogen atom and which have a strong inhibitory activity on TAFIa.

For instance, a compound of formula (I) or (II)

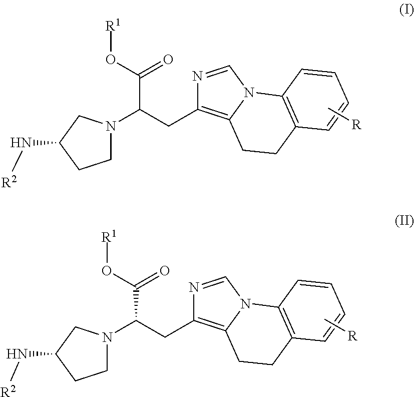

wherein either R¹ or R² or both are other than a hydrogen atom is converted to a 2-[(3S)-3-aminopyrrolidin-1-yl]-3-(4,5-dihydroimidazo[1,5-a]quinolin-3-yl)propanoic acid derivative that has the structure represented by the following formula (VI) or (VII) (where R is as defined in connection with formula (I) or (II)) and which has a strong inhibitory activity on TAFIa:

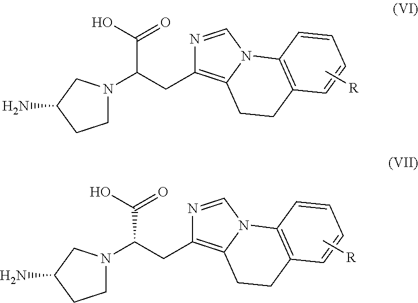

Thus, the above-described ester derivative and carbamate derivative which function as prodrugs are extremely useful compounds.

The compounds of the present invention sometimes have an asymmetric center and in that case they occur as various optical isomers or with various configurations. Hence, the compounds of the present invention may be able to occur as separate optically active substances with the (R) and (S) configurations; alternatively, they may be able to occur as a racemate or an (RS) mixture. In the case of a compound having two or more asymmetric centers, diastereomers can also occur on account of the optical isomerism of each asymmetric center. The compounds of the present invention include ones that contain all of these forms in desired proportions. For example, diastereomers can be separated by methods well known to those in the art, say, fractional crystallization, and optically active substances can be obtained by techniques in organic chemistry that are well known for this purpose. The compounds of the present invention may contain isomers such as a cis form and a trans form. The compounds of the present invention include these isomers, as well as compounds that contain these isomers in desired proportions.

The compounds of the present invention have TAFIa inhibitory activity and can be used as therapeutics or prophylactics for diseases involving TAFIa, such as deep vein thrombosis, disseminated intravascular coagulation syndrome, pulmonary embolism, cardiogenic cerebral infarction, ischemic heart disease, sepsis, pulmonary fibrosis, respiratory distress syndrome, cerebral stroke, obstructive renal disorder, Behcet's disease, mouth cancer, obesity, tissue degeneration, preeclampsia, retinal vein occlusion, inflammatory intestinal disease, arthritis, meningococcemia, and complications of kidney transplantation. The compounds of the present invention can be administered either alone or together with pharmacologically or pharmaceutically acceptable carriers or diluents. If the compounds of the present invention are to be used typically as TAFIa inhibitors, they may be administered as such either orally or parenterally. If desired, the compounds of the present invention may be administered orally or parenterally as formulations that contain them as an active ingredient. An example of the parenteral administration is intravenous administration by injection.

Since the compounds of the present invention have TAFIa inhibitory activity, patients who are suspected of the development of thrombotic diseases such as deep vein thrombosis caused by risk factors including a surgical operation such as artificial joint replacement, as well as pulmonary embolism, cardiogenic cerebral infarction and ischemic heart disease, or patients in whom the manifestation of such diseases has been confirmed may be administered with these compounds as antithrombotics or fibrinolysis promoters to prevent or treat those diseases.

In addition, the compounds of the present invention are capable of potentiating the action of tissue plasminogen activator (t-PA) and can be used in combination with t-PA preparations or they may be formulated as a combination drug in which they function as an auxiliary agent for t-PA.

The compounds of the present invention may be administered in amounts of, say, 1 mg to 1000 mg, preferably 10 mg to 200 mg, per dose, and the frequency of administration may be once to three times a day. The dosage of the compounds of the present invention can be adjusted as appropriate for the age, body weight, and symptoms of the patient under treatment.

The compounds of the present invention can be evaluated for their TAFIa activity by known procedures, such as the method described in the test procedures described hereinafter.

The methods of producing the compounds according to the present invention are hereinafter described in detail but they are not particularly limited to the examples shown below. The solvents to be used in reactions may be of any kinds that do not interfere with the respective reactions and they are not particularly limited to the following description.

Production Method 1

The compound (I) of the present invention, in which R is a hydrogen atom or a C₁₋₁₀ alkyl group, R¹ is a hydrogen atom, and R² is a hydrogen atom can be synthesized by the following method (scheme 1).

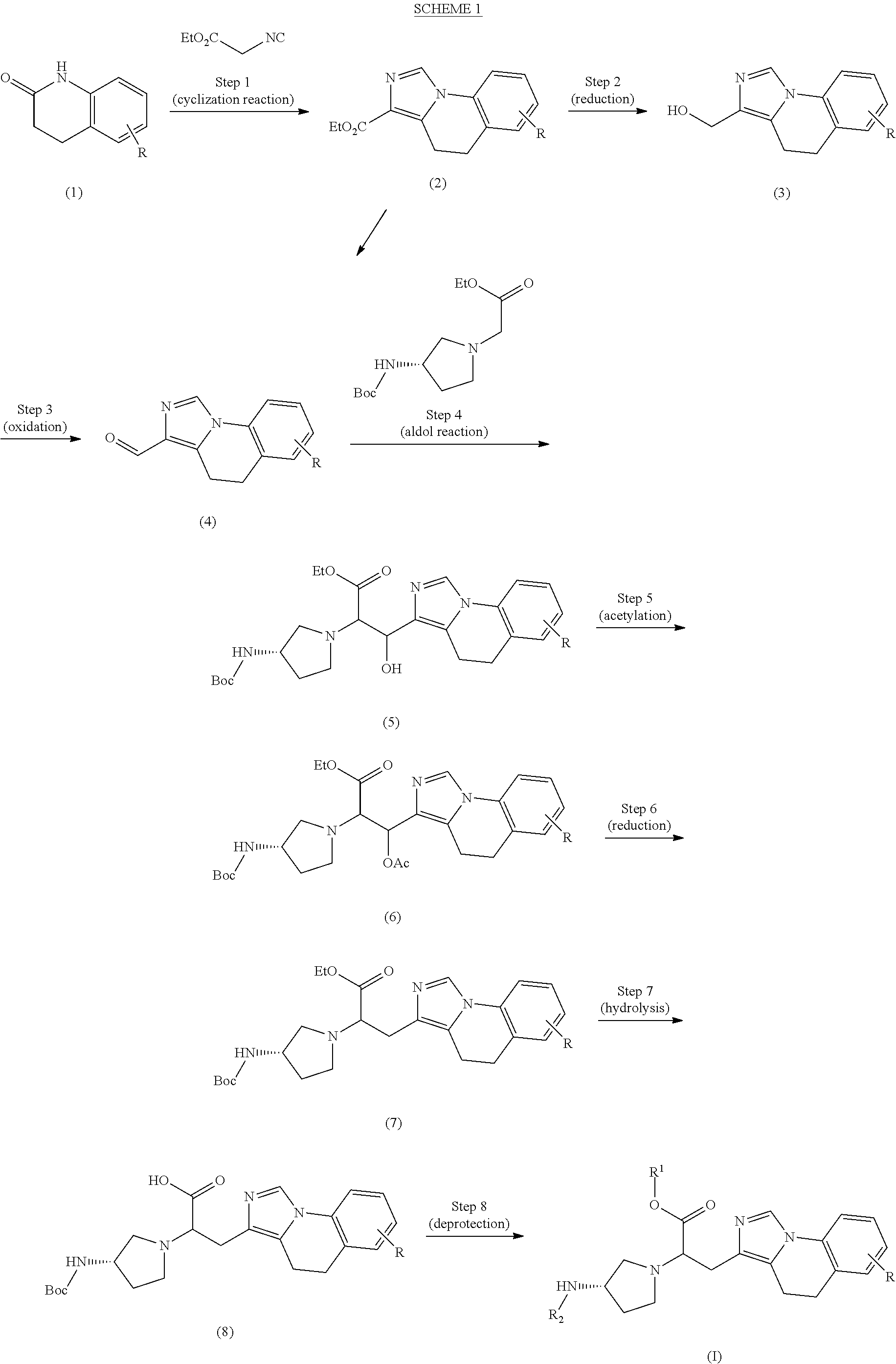

### (1) Step 1 (Cyclization Reaction)

Compound (1) is reacted with a suitable amide activator such as diethyl chlorophosphate in the presence of a suitable base to give an intermediate in the reaction system. The intermediate is reacted with ethyl isocyanoacetate in the presence of a suitable base to give compound (2). The bases to be used in this step include potassium tert-butoxide, sodium hydride, -butyllithium, lithium diisopropylamide, lithium hexamethyldisilazane, etc. The solvents to be used in the reactions include tetrahydrofuran, diethyl ether, dioxane, toluene, etc.; the reactions can be carried out at temperatures ranging from −78° C. to room temperature.

### (2) Step 2 (Reduction)

Compound (2) is reduced with a reducing agent such as lithium aluminum hydride to give compound (3). The solvents to be used in this reaction include tetrahydrofuran, diethyl ether, dioxane, toluene, etc. The reaction can be carried out at temperatures ranging from −78° C. to room temperature. Alternatively, compound (2) can be reduced with a reducing agent such as diisobutyl aluminum hydride, diisopropyl aluminum hydride, etc. to give compound (4). The solvents to be used in this reaction include tetrahydrofuran, diethyl ether, dioxane, toluene, dichloromethane, chloroform, etc.; the reactions can be carried out at temperatures ranging from −78° C. to room temperature.

### (3) Step 3 (Oxidation)

Compound (3) is reacted with a suitable oxidizing agent, optionally using a suitable base such as triethylamine or diisopropylethylamine to give compound (4). The oxidizing agents to be used in this step include dimethyl sulfoxide-oxalyl chloride, dimethyl sulfoxide-N,N′-dicyclohexylcarbodiimide (DCC), dimethyl sulfoxide-1-chloropyrrolidine-2,5-dione (NCS), dimethyl sulofixde-acetic anhydride, manganese dioxide, Dess-Martin periodinane, piridinium chlorochromate (PCC), piridinium dichromate (PDC), etc. The solvents to be used in this reaction include dichloromethane, chloroform, 1,2-dichloroethane, tetrahydrofuran, diethyl ether, dioxane, toluene, etc.; the reaction can be carried out at temperatures ranging from −78° C. to room temperature.

### (4) Step 4 (Aldol Reaction)

Compound (4) is reacted with ethyl {(3S)-3-[(tert-butoxycarbonyl)amino]pyrrolidin-1-yl}acetate in the presence of a suitable base to give compound (5). The bases to be used in this step include potassium tert-butoxide, sodium hydride, n-butyllithium, lithium diisopropylamide, lithium hexamethyldisilazane, etc. The solvents to be used in the reaction include tetrahydrofuran, diethyl ether, dioxane, toluene, etc.; the reactions can be carried out at temperatures ranging from −78° C. to room temperature.

### (5) Step 5 (Acetylation)

Compound (5) is reacted with a suitable acetylating agent using a suitable base to give compound (6). The acetylating agents to be used in this step include acetic anhydride, acetyl chloride, etc. The bases to be used in the reaction include pyridine, triethylamine, diisopropylethylamine, etc. The solvents to be used in this reaction include dichloromethane, chloroform, 1,2-dichloroethane, tetrahydrofuran, toluene, etc.; the reaction can be carried out at temperatures ranging from 0° C. to room temperature.

### (6) Step 6 (Reduction)

Compound (6) is catalytically hydrogenated in a hydrogen atmosphere using a catalyst such as palladium-activated carbon, palladium hydroxide, or platinum-activated carbon to give compound (7). The solvents to be used in this reaction include methanol, ethanol, isopropanol, ethyl acetate, tetrahydrofuran, acetic acid, mixtures thereof, etc; the reaction can be carried out at temperatures ranging from room temperature to the reflux temperature.

### (7) Step 7 (Hydrolysis)

Compound (7) is hydrolyzed with a suitable base to give compound (8). The bases to be used in this step include lithium hydroxide, sodium hydroxide, and potassium hydroxide. The solvents to be used in this reaction include methanol, ethanol, isopropanol, tetrahydrofuran, water, mixtures thereof, etc; the reaction can be carried out at temperatures ranging from 0° C. to the reflux temperature.

### (8) Step 8 (Deprotection)

Compound (8) is deprotected with a suitable acid to give the compound (I) of the present invention. The suitable acids to be used in this step include hydrochloric acid, sulfuric acid, hydrobromic acid, trifluoroacetic acid, methanesulfonic acid, benzenesulfonic acid, p-toluenesulfonic acid, 10-camphorsulfonic acid, etc. The solvents to be used in this reaction include chloroform, dichloromethane, methanol, ethanol, isopropanol, ethyl acetate, tetrahydrofuran, diethyl ether, dioxane, toluene, water, etc.; the reaction can be carried out at temperatures ranging from 0° C. to the reflux temperature.

Production Method 2

The compound (I) of the present invention, in which R is a hydrogen atom or a C₁₋₁₀ alkyl group, R¹ is a hydrogen atom, and R² is a hydrogen atom can also be synthesized by the following method (scheme 2).

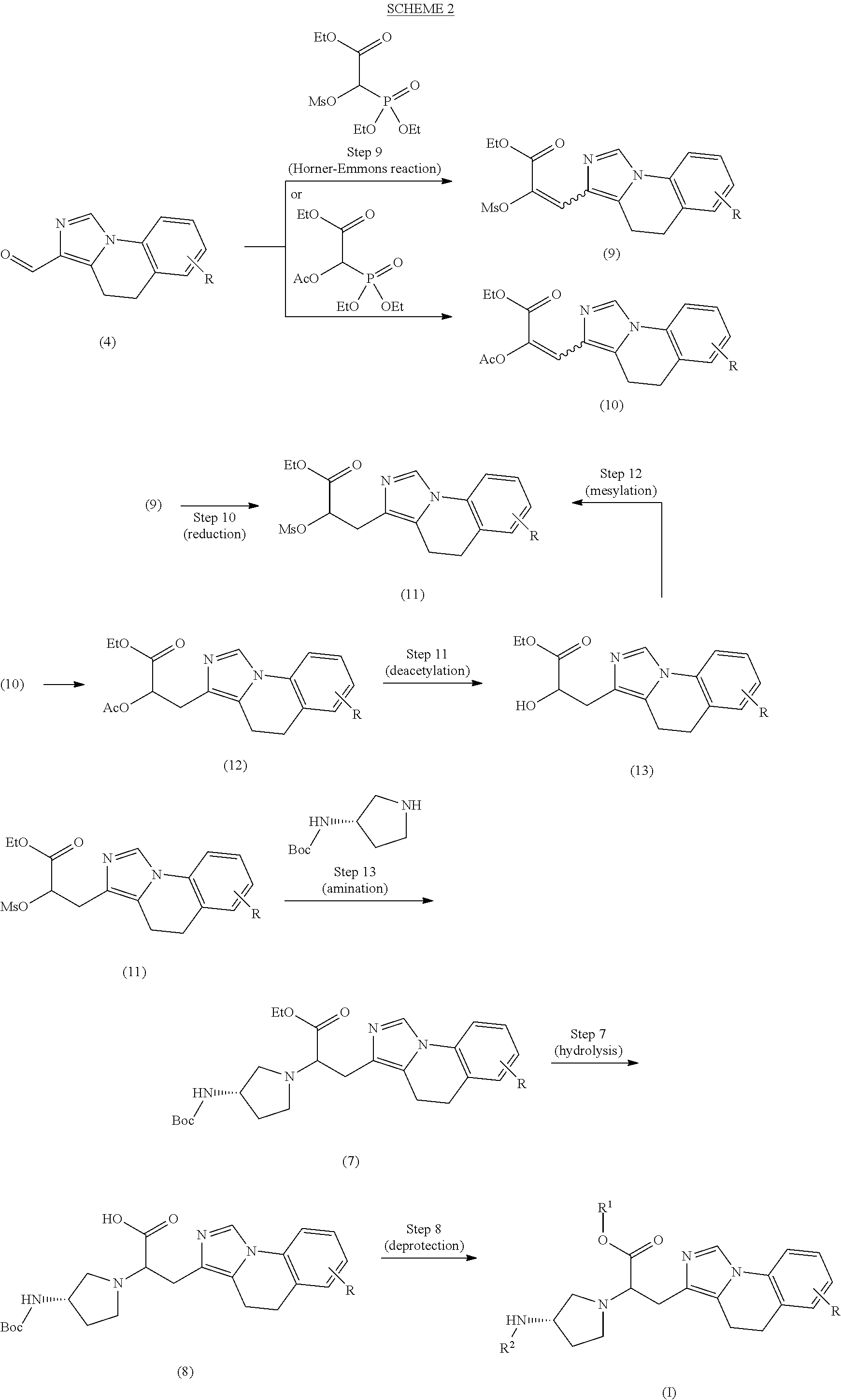

### (9) Step 9 (Horner-Emmons Reaction)

Compound (4) is reacted with a suitable Horner-Emmons reagent such as ethyl (diethoxyphosphoryl)(methylsulfonyloxy)acetate or ethyl (acetoxy)(diethoxyphosphoryl)acetate in the presence of a suitable base and in the presence or absence of a metal halide such as lithium chloride to give compound (9) or (10). The bases to be used in this step include 1,1,3,3-tetramethylguanidine, diisopropylethylamine, potassium tert-butoxide, sodium hydride, n-butyllithium, lithium diisopropylamide, lithium hexamethyldisilazane, sodium hexamethyldisilazane, etc. The solvents to be used in the reaction include tetrahydrofuran, diethylether, dioxane, toluene, etc.; the reaction can be carried out at temperatures ranging from −78° C. to room temperature.

### (10) Step 10 (Reduction)

Compound (9) or (10) is catalytically hydrogenated in a hydrogen atmosphere using a catalyst such as palladium-activated carbon, palladium hydroxide, or platinum-activated carbon to give compound (11) or (12). The solvents to be used in this reaction include methanol, ethanol, isopropanol, ethyl acetate, tetrahydrofuran, acetic acid, mixtures thereof, etc; the reaction can be carried out at temperatures ranging from room temperature to the reflux temperature.

### (11) Step 11 (Deacetylation)

Compound (12) is reacted with a suitable base such as potassium carbonate, sodium ethoxide, etc. to give compound (13). The solvents to be used in this reaction include ethanol, tetrahydrofuran, diethyl ether, dioxane, toluene, etc.; the reaction can be carried out at temperatures ranging from 0° C. to room temperature.

### (12) Step 12 (Mesylation)

Compound (13) is reacted with methanesulfonyl chloride in the presence of a suitable base to give compound (11). The bases to be used in this step include potassium carbonate, cesium carbonate, triethylamine, diisopropylethylamine, pyridine, 4-dimethylaminopyridine, etc. The solvents to be used in this reaction include tetrahydrofuran, diethyl ether, dioxane, toluene, dichloromethane, chloroform, acetonitrile, etc.; the reaction can be carried out at temperatures ranging from 0° C. to room temperature.

### (13) Step 13 (Amination)

Compound (11) is reacted with tert-butyl (3S)-pyrrolidin-3-yl-carbamate in the presence of a suitable base to give compound (7). The bases to be used in this step include triethylamine, diisopropylethylamine, pyridine, 4-dimethylaminopyridine, etc. The solvents to be used in this reaction include tetrahydrofuran, diethyl ether, dioxane, toluene, dichloromethane, chloroform, acetonitrile, N,N-dimethylformamide, etc.; the reaction can be carried out at temperatures ranging from 0° C. to the reflux temperature.

From compound (7), the compound (I) of the present invention can be synthesized by the same procedures as steps 7 and 8 described in production method 1.

Production Method 3

The compound (I) of the present invention, in which R is a hydrogen atom or a C₁₋₁₀ alkyl group, R¹ is a hydrogen atom, and R² is a hydrogen atom can also be synthesized by the following method (scheme 3).

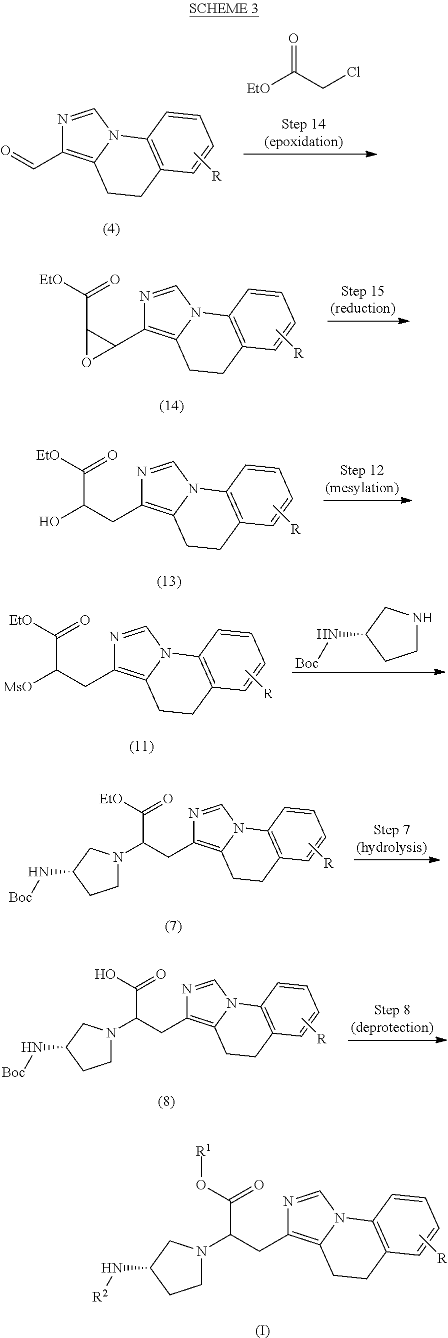

### (14) Step 14 (Epoxidation)

Compound (4) is reacted with ethyl chloroacetate in the presence of a suitable base to give compound (14). The bases to be used in this step include sodium ethoxide, sodium methoxide, potassium tert-butoxide, sodium hydride, n-butyllithium, lithium diisopropylamide, lithium hexamethyldisilazane, sodium hexamethyldisilazane, etc. The solvents to be used in this reaction include tetrahydrofuran, diethyl ether, dioxane, toluene, etc.; the reaction can be carried out at temperatures ranging from −78° C. to the reflux temperature.

### (15) Step 15 (Reduction)

Compound (14) is catalytically hydrogenated in a hydrogen atmosphere using a catalyst such as palladium-activated carbon, palladium hydroxide, or platinum-activated carbon to give compound (13). The solvents to be used in this reaction include ethanol, ethyl acetate, tetrahydrofuran, mixtures thereof, etc; the reaction can be carried out at temperatures ranging from room temperature to the reflux temperature.

From compound (13), the compound (I) of the present invention can be synthesized via four steps in production method 2, i.e., step 12, step 13, step 7 and step 8, by taking the same procedures.

Production Method 4

The compound (II) of the present invention, in which R is a hydrogen atom or a C₁₋₁₀ alkyl group, R¹ is a hydrogen atom, and R² is a hydrogen atom can be synthesized by the following method (scheme 4).

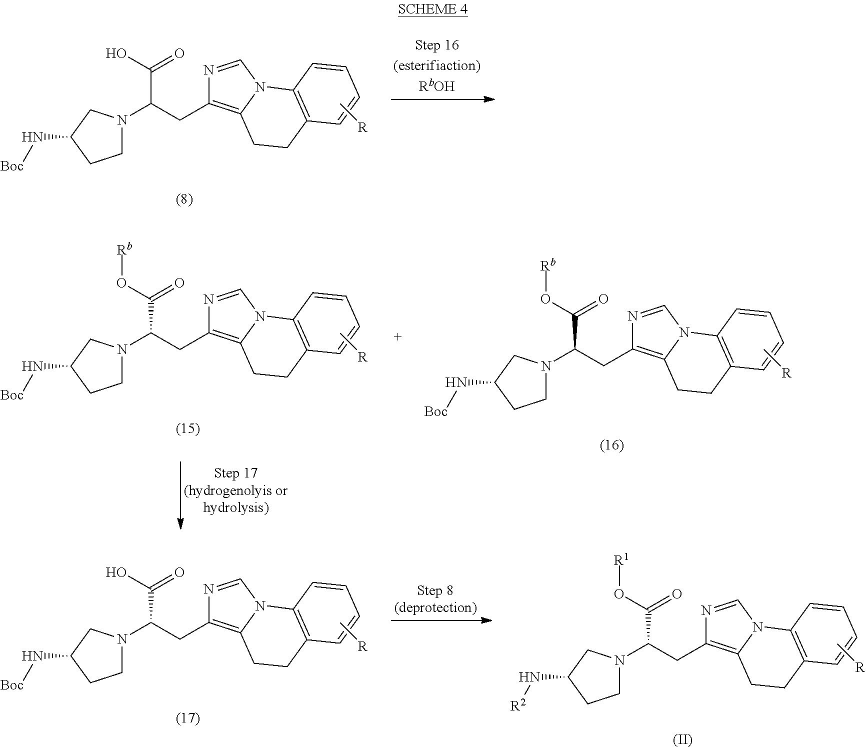

### (15) Step 16 (Esterification)

Compound (8) is reacted with a chiral alcohol (R^{b}OH) such as (1R,2S)-1-phenyl-2-(pyrrolidin-1-yl)propan-1-ol, (1R)-1-phenylethanol, (1S,2R,5S)-5-methyl-2-(propan-2-yl)cyclohexanol, or (3R)-3-hydroxy-4,4-dimethyldihydrofuran-2(3H)-one using a condensing agent in the presence or absence of a suitable base to give compound (15) and compound (16) as diastereomers that are separable by silica gel column chromatography. Suitable bases include triethylamine, diisopropylamine, pyridine, 4-dimethylaminopyridine, etc. The condensing agents to be used in this step include N-[3-(dimethylamino)propyl]-N′-ethylcarbodiimide hydrochloride, N,N′-dicyclohexylcarbodiimide, di-1H-imidazol-1-yl-methanone, etc. The solvents to be used in the reaction include dichloromethane, chloroform, 1,2-dichloroethane, tetrahydrofuran, toluene, N,N-dimethylformamide, etc.; the reaction can be carried out at temperatures ranging from 0° C. to the reflux temperature.

### (17) Step 17 (Hydrogenolysis or Hydrolysis)

Compound (15) is catalytically hydrogenated in a hydrogen atmosphere using a catalyst such as palladium-activated carbon, palladium hydroxide, or platinum-activated carbon to give compound (17). The solvents to be used in this reaction include methanol, ethanol, isopropanol, ethyl acetate, tetrahydrofuran, mixtures thereof, etc; the reaction can be carried out at temperatures ranging from room temperature to the reflux temperature. Alternatively, compound (15) is hydrolyzed using a suitable base to give compound (17). The bases to be used in this step include lithium hydroxide, sodium hydroxide, potassium hydroxide, etc. The solvents to be used in this reaction include methanol, ethanol, isopropanol, tetrahydrofuran, water, mixtures thereof, etc; the reaction can be carried out at temperatures ranging from 0° C. to the reflux temperature.

From compound (17), the compound (II) of the present invention can be synthesized by the same procedure as step 8 described in production method 1.

Production Method 5

The compound (II) of the present invention, in which R is a hydrogen atom or a C₁₋₁₀ alkyl group, R¹ is a C₁₋₁₀ alkyl group, a C₃₋₈ cycloalkyl group, or a substituent having the structure represented by the following formula Ia or Ib:

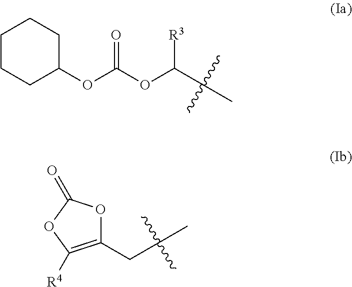

where R³ is a C₁₋₆ alkyl group and R⁴ is a C₁₋₆ alkyl group, a C₃₋₈ cycloalkyl group, or a benzyl group, and R² is a hydrogen atom can be synthesized by the following method (scheme 5).

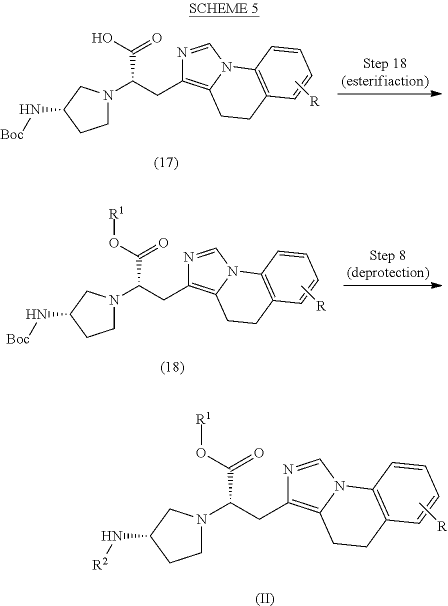

### (18) Step 18 (Esterification)

Compound (17) is reacted with an alcohol such as methanol, ethanol or propanol using a condensing agent in the presence or absence of a suitable base to give compound (18). Suitable bases include triethylamine, diisopropylamine, pyridine, 4-dimethylaminopyridine, etc. The condensing agents to be used in this step include N-[3-(dimethylamino)propyl]-N′-ethylcarbodiimide hydrochloride, N,N′-dicyclohexylcarbodiimide, di-1H-imidazol-1-yl-methanone, etc. The solvents to be used in the reaction include dichloromethane, chloroform, 1,2-dichloroethane, tetrahydrofuran, toluene, N,N-dimethylformamide, etc.; the reaction can be carried out at temperatures ranging from 0° C. to the reflux temperature. Alternatively, compound (17) is reacted with an alkyl halide such as methyl iodide, ethyl iodide or propyl iodide in the presence of a suitable base to give compound (18). Suitable bases include potassium carbonate, cesium carbonate, etc. The solvents to be used in this reaction include tetrahydrofuran, toluene, N,N-dimethylformamide, acetone, etc.; the reaction can be carried out at temperatures ranging from 0° C. to the reflux temperature. As a further approach, compound (17) is reacted with an alcohol such as methanol, ethanol or propanol using a suitable azo reagent in the presence of a suitable phosphine reagent to give compound (18). Suitable phosphine reagents include triphenylphosphine, tri-n-butylphosphine, tri-tert-butylphosphine, etc. Suitable azo reagents include diethyl azodicarboxylate, diisopropyl azodicarboxylate, tetramethyl azodicarboxamide, azodicarbonyl dipiperidine, etc.

From compound (18), the compound (II) of the present invention can be synthesized by the same procedures as step 8 described in production method 1.

Production Method 6

The compound (II) of the present invention, in which R is a hydrogen atom or a C₁₋₁₀ alkyl group, R¹ is a hydrogen atom, and R² is a substituent having the structure represented by the following formula Ic or Id:

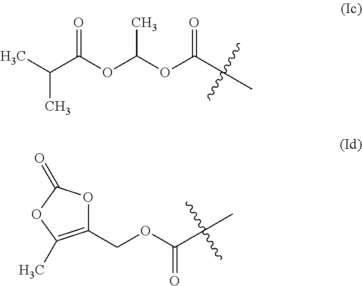

can be synthesized by the following method (scheme 6).

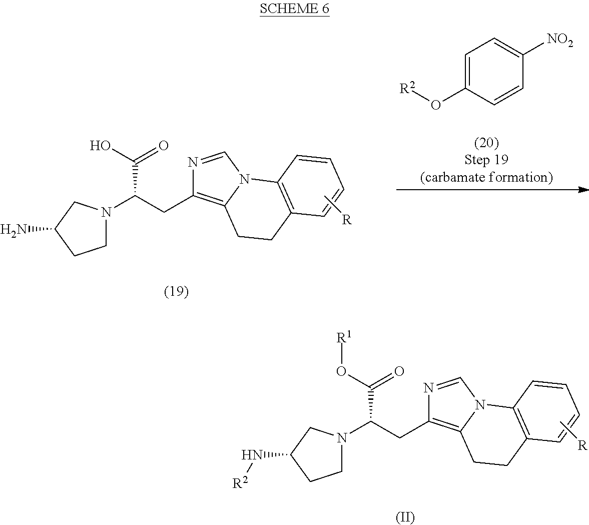

### (19) Step 19 (Carbamate Formation)

Compound (19) is reacted with active carbonate (20) to give the compound (II) of the present invention. The solvents to be used in this reaction include N,N-dimethylformamide, N,N-dimethyl acetamide, N-methylpyrrolidone, tetrahydrofuran, toluene, dichloromethane, chloroform, water, etc.; the reaction can be carried out at temperatures ranging from 0° C. to the reflux temperature.

Production Method 7

The compound (II) of the present invention, in which R is a hydrogen atom or a C₁₋₁₀ alkyl group; R¹ is a C₁₋₁₀ alkyl group, a C₃₋₈ cycloalkyl group or a substituent having the structure represented by the following formula Ia or Ib:

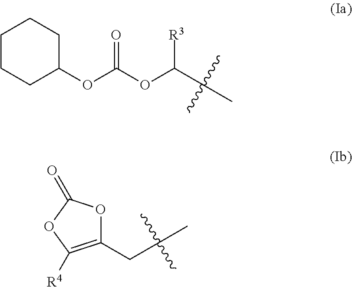

where R³ is a C₁₋₆ alkyl group;
R⁴ is a C₁₋₆ alkyl group, a C₃₋₈ cycloalkyl group, or a benzyl group; and
R² is a substituent having the structure represented by the following formula Ic or Id:

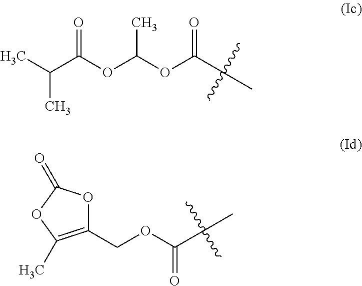

can be synthesized by the following method (scheme 7).

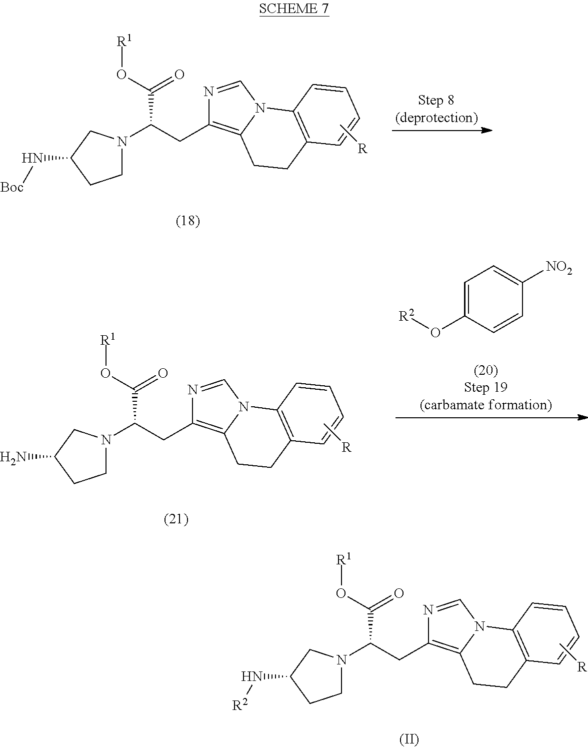

From compound (18), the compound (II) of the present invention can be synthesized via step 8 in production method 1 and via step 19 in production method 6 by taking the same procedures.

On the following pages, the present invention is described even more specifically by showing Reference Examples, Examples of the invention, and Tests.

### EXAMPLES

The NH silica gel column chromatography as referred to in the following Examples means purification by column chromatographic separation using an NH2 type silica gel (Chromatorex NH2 type; FUJI SILYSIA CHEMICAL LTD.) The diol silica gel column chromatography means purification by column chromatographic separation using a diol type silica gel (Purif-Pack DIOL-60 μm; Shoko Scientific Co., Ltd.) The optical purities of compounds of the present invention were calculated based on measurements under the following conditions:

### Column: CHIRALPACK AD-3, 4Φ×250 mm, 3 μm (DAICEL CHEMICAL INDUSTRIES, LTD.)

Column temperature: 10° C.
Flow rate: 1.0 mL/min

### Detection: UV, 240 nm

Sample concentration: 1 mg/mL
Injection volume: 2 μL
Mobile phase: n-Hexane:IPA:TFA:DEA=85:15:0.5:0.5

### Reference Example 1

### Synthesis of 4-(bromomethyl)-5-tert-butyl-1,3-dioxol-2-one

(1) Synthesis of benzyl 4,4-dimethyl-3-oxopentanoate

To a solution of methyl 4,4-dimethyl-3-oxopentanoate (8.01 g) in toluene (150 ml), benzyl alcohol (12.14 g) and lithium perchlorate (1.09 g) were added and the mixture was heated under reflux for 5 hours. After removing the solvent in vacuo, the resulting residue was purified by silica gel column chromatography (eluent; n-hexane/ethyl acetate=9:1) to give the titled compound (9.64 g) as a pale yellow oil.

MS (ESI/APCI Dual) m/z 235 [M+H]⁺

(2) Synthesis of [1-(benzyloxy)-4,4-dimethyl-1,3-dioxopentan-2-yl]diazen-2-ium-1-ide

To a solution of benzyl 4,4-dimethyl-3-oxopentanoate (9.63 g) in acetonitrile (200 ml), triethylamine (17.3 ml) and 4-(acetylamino)benzenesulfonyl azide (9.88 g) were added in small portions under cooling with ice and the mixture was stirred for an hour at the same temperature and for an additional three hours at room temperature. The precipitating crystal was recovered by filtration and the filtrate was concentrated. The resulting residue was purified by silica gel column chromatography (eluent; n-hexane/ethyl acetate=19:1 to 9:1) to give the titled compound (8.37 g) as a yellow oil.

MS (ESI/APCI Dual) m/z 261 [M+H]⁺

(3) Synthesis of benzyl 2-hydroxy-4,4-dimethyl-3-oxopentanoate

To a solution of [1-(benzyloxy)-4,4-dimethyl-1,3-dioxopentan-2-yl]diazen-2-ium-1-ide (8.36 g) in a 2:1 mixture (150 ml) of tetrahydrofuran and water, rhodium(II) acetate (dimer) (75 mg) was added and the resulting mixture was stirred for 3 hours at 90° C. Rhodium(II) acetate (dimer) (79 mg) was then added and the resulting mixture was stirred for an additional four hours at 90° C. Rhodium(II) acetate (dimer) (281 mg) was further added and the resulting mixture was stirred for an additional five hours at 90° C. The solvents were removed in vacuo and after adding brine, extracting with ethyl acetate was performed twice. After drying the combined organic layers with anhydrous magnesium sulfate, the desiccant was filtered off and the solvents were removed in vacuo. The resulting residue was purified, first by silica gel column chromatography (eluent; n-hexane/ethyl acetate=85:15), then by NH silica gel column chromatography (eluent; n-hexane/ethyl acetate=9:1) to give the titled compound (3.45 g) as a yellow oil.

MS (ESI/APCI Dual) m/z 273 [M+H]⁺

(4) Synthesis of benzyl 5-tert-butyl-2-oxo-1,3-dioxol-4-carboxylate

To a solution of benzyl 2-hydroxy-4,4-dimethyl-3-oxopentanoate (3.44 g) in tetrahydrofuran (70 ml), diisopropylethylamine (179 mg) and di-1H-imidazol-1-yl-methanone (4.44 g) were added under cooling with ice and the mixture was stirred for an hour at the same temperature and for an additional 5.5 hours at room temperature. To the reaction mixture, an aqueous solution of 1 M HCl was added and extracting with ethyl acetate was performed twice. After washing the combined organic layers with brine and drying the same with anhydrous magnesium sulfate, the desiccant was filtered off and the solvents were removed in vacuo. The resulting residue was washed with n-hexane to give the titled compound (1.28 g) as a colorless powder.

MS (EI) m/z 276 [M]⁺

(5) Synthesis of 5-tert-butyl-2-oxo-1,3-dioxol-4-carboxylic acid

To a solution of benzyl 5-tert-butyl-2-oxo-1,3-dioxol-4-carboxylate (2.52 g) in ethanol (45 ml), 20% palladium hydroxide (50% hydrous; 129 mg) was added and the mixture was stirred for an hour at room temperature under hydrogen purge. The reaction mixture was filtered through Celite and the solvent was removed in vacuo to give the titled compound (1.71 g) as an unpurified colorless powder.

MS (ESI/APCI Dual) m/z 185 [M−H]⁻

(6) Synthesis of 4-tert-butyl-5-(hydroxymethyl)-1,3-dioxol-2-one

To a solution of 5-tert-butyl-2-oxo-1,3-dioxol-4-carboxylic acid (1.70 g) in chloroform (50 ml), N,N-dimethylformamide (70 μl) and oxalyl chloride (0.88 ml) were added dropwise under cooling with ice and the mixture was stirred for 30 minutes at the same temperature and for an additional hour at room temperature. The solvent was removed in vacuo and after adding chloroform (45 ml) to the resulting residue, the mixture was cooled to −60° C. A solution of tetrabutylammonium borohydride (2.60 g) in chloroform (15 ml) was added dropwise and the mixture was stirred for 1.5 hours at the same temperature. To the reaction mixture, an aqueous solution of 1 M HCl was added and the mixture was left to stand until room temperature was reached; thereafter, brine and chloroform were added to cause liquid separation. After extracting the aqueous layer with chloroform, the organic layers were combined and dried over anhydrous magnesium sulfate; thereafter, the desiccant was filtered off and the solvent was removed in vacuo. The resulting residue was purified by silica gel column chromatography (eluent; n-hexane/ethyl acetate=3:2) to give the titled compound (854 mg) as a colorless oil.

MS (EI) m/z 172 [M]⁺

(7) Synthesis of 4-(bromomethyl)-5-tert-butyl-1,3-dioxol-2-one

To a solution of 4-tert-butyl-5-(hydroxymethyl)-1,3-dioxol-2-one (406 mg) in chloroform (5 ml), carbon tetrabromide (943 mg) and triphenylphosphine (750 mg) were added and the mixture was stirred for 18 hours at room temperature. The solvent was removed in vacuo and the resulting residue was purified by silica gel column chromatography (eluent; n-hexane/ethyl acetate=85:15) to give the titled compound (490 mg) as a colorless oil.

MS (EI) m/z 234 [M]⁺

¹H NMR (300 MHz, CHLOROFORM-d) δ ppm 1.32 (s, 9H), 4.29 (s, 2H)

### Reference Example 2

### Synthesis of 4-(bromomethyl)-5-(2-methylpropyl)-1,3-dioxol-2-one

Using ethyl 5-methyl-3-oxohexanoate instead of methyl 4,4-dimethyl-3-oxopentanoate, the procedures of (1) to (7) in Reference Example 1 were repeated to give the titled compound.

MS (EI) m/z 234 [M]⁺

¹H NMR (300 MHz, CHLOROFORM-d) δ ppm 0.99 (d, J=6.7 Hz, 6H), 1.92-2.08 (m, 1H), 2.31 (d, J=7.0 Hz, 2H), 4.18 (s, 2H)

### Reference Example 3

### Synthesis of 4-(bromomethyl)-5-cyclohexyl-1,3-dioxol-2-one

Using ethyl 3-cyclohexyl-3-oxopropanoate instead of methyl 4,4-dimethyl-3-oxopentanoate, the procedures of (1) to (7) in Reference Example 1 were repeated to give the titled compound.

¹H NMR (300 MHz, CHLOROFORM-d) δ ppm 1.16-1.57 (m, 5H), 1.65-1.79 (m, 1H), 1.80-1.93 (m, 4H), 2.40-2.57 (m, 1H), 4.22 (s, 2H)

### Reference Example 4

### Synthesis of 4-benzyl-5-(bromomethyl)-1,3-dioxol-2-one

Using ethyl 3-oxo-4-phenylbutanoate instead of methyl 4,4-dimethyl-3-oxopentanoate, the procedures of (1) to (7) in Reference Example 1 were repeated to give the titled compound.

MS (EI) m/z 268[M]⁺

¹H NMR (300 MHz, CHLOROFORM-d) δ ppm 3.78 (s, 2H), 3.97 (s, 2H), 7.25-7.42 (m, 5H)

### Example 1

### Synthesis of (2S)-2-[(3S)-3-aminopyrrolidin-1-yl]-3-(4,5-dihydroimidazo[1,5-a]quinolin-3-yl)propanoic acid trihydrochloride

(1) Synthesis of ethyl 4,5-dihydroimidazo[1,5-a]quinoline-3-carboxylate

To a solution of 3,4-dihydroquinolin-2(1H)-one (50 g) in tetrahydrofuran (1 L), potassium tert-butoxide (46 g) was added under cooling with ice and the mixture was stirred for 30 minutes at the same temperature. Diethyl chlorophosphate (70 g) was added and after stirring the mixture for 30 minutes at the same temperature, ethyl isocyanoacetate (31 g) and potassium tert-butoxide (46 g) were added at −30° C. and the mixture was stirred for an hour at room temperature. To the reaction mixture, an aqueous solution of 15% citric acid was added and extracting with ethyl acetate and washing with brine were performed. After drying over anhydrous sodium sulfate, the desiccant was filtered off and the solvents were removed in vacuo. The resulting residue was purified by silica gel column chromatography (eluent; n-hexane/ethyl acetate=1:1 to 1:3) to give the titled compound (64.4 g) as a brown powder.

MS (ESI/APCI Dual) m/z 243 [M+H]⁺

¹H NMR (300 MHz, CHLOROFORM-d) δ ppm 1.43 (t, J=7.2 Hz, 3H), 2.96 (t, J=7.2 Hz, 2H), 3.35 (t, J=7.2 Hz, 2H), 4.41 (q, J=7.2 Hz, 2H), 7.20-7.30 (m, 1H), 7.30-7.41 (m, 2H), 7.42-7.52 (m, 1H), 8.03 (s, 1H)

(2) Synthesis of 4,5-dihydroimidazo[1,5-a]quinolin-3-yl-methanol

To a solution of ethyl 4,5-dihydroimidazo[1,5-a]quinoline-3-carboxylate (56.4 g) in tetrahydrofuran (583 ml), lithium aluminum hydride (10.6 g) was added under cooling with ice and the mixture was stirred for an hour at the same temperature. Ethy acetate and water were added to the reaction system and the mixture was filtered; brine was added to the filtrate and the mixture was subjected to extraction with chloroform. After drying over anhydrous sodium sulfate, the desiccant was filtered off and the solvents were removed in vacuo to give the titled compound (50.1 g) as a brown oil.

¹H NMR (300 MHz, CHLOROFORM-d) δ ppm 2.84-3.07 (m, 4H), 4.64 (s, 2H), 7.14-7.25 (m, 1H), 7.27-7.37 (m, 2H), 7.40-7.45 (m, 1H), 8.00 (s, 1H)

(3) Synthesis of 4,5-dihydroimidazo[1,5-a]quinoline-3-carbaldehyde

To a solution of 4,5-dihydroimidazo[1,5-a]quinolin-3-yl-methanol (50.1 g) in chloroform (777 ml), manganese dioxide (101 g) was added and the mixture was stirred for 15 hours at room temperature. The reaction system was filtered through Celite and the solvent was removed in vacuo. The resulting powder was washed with 1:1 n-hexane/ethyl acetate to give the titled compound (20 g) as a light brown powder.

MS (ESI/APCI Dual) m/z 199 [M+H]⁺

¹H NMR (300 MHz, CHLOROFORM-d) δ ppm 2.97 (t, J=7.2 Hz, 2H), 3.35 (t, J=7.2 Hz, 2H), 7.21-7.31 (m, 1H), 7.31-7.42 (m, 2H), 7.42-7.54 (m, 1H), 8.07 (s, 1H), 10.02 (s, 1H)

(4) Synthesis of ethyl (2Z)-2-(acetoxy)-3-(4,5-dihydroimidazo[1,5-a]quinolin-3-yl)-2-propenoate and ethyl (2E)-2-(acetoxy)-3-(4,5-dihydroimidazo[1,5-a]quinolin-3-yl)-2-propenoate

To a solution of ethyl (acetoxy)(diethoxyphosphoryl)acetate (26.7 g) in tetrahydrofuran (200 ml), lithium chloride (3.99 g) was added, followed by dropwise addition of 1,1,3,3-tetramethylguanidine (11.0 g) at −78° C. and the mixture was stirred for 25 minutes at the same temperature. Subsequently, a solution of 4,5-dihydroimidazo[1,5-a]quinoline-3-carbaldehyde (14.4 g) in tetrahydrofuran (800 ml) was added dropwise and the mixture was stirred for 30 minutes at the same temperature. After stirring the mixture for an additional 1.5 hours at room temperature, a saturated aqueous solution of ammonium chloride (200 ml) was added under cooling with ice to quench the reaction. Water (500 ml) was added to separate the organic layer, which was then concentrated. The residue was dissolved in chloroform (1000 ml); after extracting the previously obtained aqueous layer, the organic layer was washed with brine. After drying over anhydrous magnesium sulfate, the desiccant was filtered off and the solvents were removed in vacuo to give the titled compound (34.0 g; a mixture of E and Z forms) as an unpurified orange oil. A 3.38 g (ca. 10%) portion of the oil was purified by silica gel column chromatography (eluent: chloroform/ethyl acetate=80:20 to 50:50), then by another run of silica gel column chromatography (eluent: n-hexane/ethyl acetate=65:35 to 20:80); the resulting powder was washed with n-hexane to give the two titled compounds as a colorless powder, i.e., the low-polarity compound ethyl (2Z)-2-(acetoxy)-3-(4,5-dihydroimidazo[1,5-a]quinolin-3-yl)-2-propenoate (588 mg) and the high-polarity compound ethyl (2E)-2-(acetoxy)-3-(4,5-dihydroimidazo[1,5-a]quinolin-3-yl)-2-propenoate (1.22 g).

Ethyl (2Z)-2-(acetoxy)-3-(4,5-dihydroimidazo[1,5-a]quinolin-3-yl)-2-propenoate

MS (ESI/APCI Dual) m/z 327 [M+H]⁺

¹H NMR (300 MHz, CHLOROFORM-d) δ ppm 1.34 (t, J=7.1 Hz, 3H), 2.40 (s, 3H), 2.90-2.98 (m, 2H), 3.00-3.09 (m, 2H), 4.30 (q, J=7.1 Hz, 2H), 7.18-7.25 (m, 1H), 7.29-7.37 (m, 3H), 7.39-7.45 (m, 1H), 8.06 (s, 1H) Ethyl (2E)-2-(acetoxy)-3-(4,5-dihydroimidazo[1,5-a]quinolin-3-yl)-2-propenoate

MS (ESI/APCI Dual) m/z 327 [M+H]⁺

¹H NMR (300 MHz, CHLOROFORM-d) δ ppm 1.25 (t, J=7.1 Hz, 3H), 2.25 (s, 3H), 2.79-2.95 (m, 4H), 4.25 (q, J=7.1 Hz, 2H), 6.69 (s, 1H), 7.15-7.24 (m, 1H), 7.28-7.38 (m, 2H), 7.40-7.47 (m, 1H), 8.04 (s, 1H)

(5) Synthesis of ethyl 2-(acetoxy)-3-(4,5-dihydroimidazo[1,5-a]quinolin-3-yl)propanoate

The unpurified ethyl 2-(acetoxy)-3-(4,5-dihydroimidazo[1,5-a]quinolin-3-yl)-2-propenoate (30.6 g; a mixture of E and Z forms), as obtained in the previous reaction, was dissolved in 1:1 ethanol/tetrahydrofuran (200 ml); to the resulting solution, 5% palladium-activated carbon (52% hydrous; 8.1 g) was added and the mixture was stirred for 67 hours at room temperature under hydrogen purge. Another portion of palladium-activated carbon (52% hydrous; 4.0 g) was added and the mixture was stirred for 24 hours at room temperature under hydrogen purge. The reaction mixture was passed through Celite and the solvents were removed in vacuo. The resulting residue was purified by NH silica gel column chromatography (eluent: chloroform), then by silica gel column chromatography (eluent: n-hexane/ethyl acetate=1:1 to 1:3) to give the titled compound (16.5 g) as a yellow oil.

MS (ESI/APCI Dual) m/z 329 [M+H]⁺

¹H NMR (300 MHz, CHLOROFORM-d) δ ppm 1.26 (t, J=7.1 Hz, 3H), 2.09 (s, 3H), 2.90 (s, 4H), 3.05-3.21 (m, 2H), 4.21 (q, J=7.1 Hz, 2H), 5.25-5.30 (m, 1H), 7.14-7.21 (m, 1H), 7.28-7.34 (m, 2H), 7.39-7.44 (m, 1H), 7.95 (s, 1H)

(6) Synthesis of ethyl 3-(4,5-dihydroimidazo[1,5-a]quinolin-3-yl)-2-hydroxypropanoate

To a solution of ethyl 2-(acetoxy)-3-(4,5-dihydroimidazo[1,5-a]quinolin-3-yl)-2-propanoate (16.5 g) in ethanol (150 ml), sodium ethoxide (15.3 g as 20% ethanol solution) was added and the mixture was stirred for 3 hours at room temperature. The reaction mixture was evaporated under reduced pressure until its volume decreased to about a quarter of the initial value; thereafter, a saturated aqueous solution of ammonium chloride (100 ml) was added and extracting with chloroform was performed. The organic layer was washed with brine and after drying over anhydrous magnesium sulfate, the desiccant was filtered off and the solvents were removed in vacuo to give the titled compound (12.0 g) as an unpurified light brown powder.

MS (ESI/APCI Dual) m/z 287 [M+H]⁺

¹H NMR (300 MHz, CHLOROFORM-d) δ ppm 1.27 (t, J=7.1 Hz, 3H), 2.82-2.93 (m, 4H), 2.94-3.14 (m, 2H), 4.14-4.27 (m, 2H), 4.49-4.58 (m, 1H), 7.14-7.21 (m, 1H), 7.27-7.34 (m, 2H), 7.38-7.43 (m, 1H), 7.95 (s, 1H)

(7) Synthesis of ethyl 2-{(3S)-3-[(tert-butoxycarbonyl)amino]pyrrolidin-1-yl}-3-(4,5-dihydroimidazo[1,5-a]quinolin-3-yl)propanoate

To a solution of ethyl 3-(4,5-dihydroimidazo[1,5-a]quinolin-3-yl)-2-hydroxypropanoate (1.25 g) in chloroform (25 ml), methanesulfonyl chloride (0.51 ml) was added under cooling with ice, followed by dropwise addition of triethylamine (1.85 ml) and stirring for an hour at the same temperature. Brine was added to the reaction mixture and extracting with chloroform was performed. After drying over anhydrous magnesium sulfate, the desiccant was filtered off and the solvent was removed in vacuo. The residue was dissolved in chloroform (25 ml) and after adding tert-butyl (3S)-pyrrolidin-3-yl-carbamate (2.45 g) and triethylamine (2.45 ml), the mixture was stirred for 16 hours at 60° C. Brine was added to the reaction mixture and extracting with chloroform was performed. After drying over anhydrous magnesium sulfate, the desiccant was filtered off and the solvent was removed in vacuo. The residue was purified by silica gel column chromatography (eluent:ethyl acetate→chloroform/methanol=95:5), then by NH silica gel column chromatography (eluent: n-hexane/ethyl acetate=2:3 to 0:1), and again by silica gel column chromatography (eluent: ethyl acetate→chloroform/methanol=90:10) to give the titled compound (1.14 g) as a pale yellow gum.

MS (ESI/APCI Dual) m/z 455 [M+H]⁺

¹H NMR (300 MHz, CHLOROFORM-d) δ ppm 1.13-1.21 (m, 3H), 1.41, 1.43 (S, 9H), 1.54-1.70 (m, 1H), 2.08-2.26 (m, 1H), 2.58-2.79 (m, 2H), 2.82-3.11 (m, 8H), 3.67-3.74 (m, 1H), 4.00-4.20 (m, 3H), 4.95-5.23 (m, 1H), 7.12-7.20 (m, 1H), 7.26-7.33 (m, 2H), 7.38-7.43 (m, 1H), 7.94, 7.95 (S, 1H)

(8) Synthesis of (1R)-1-phenylethyl (2S)-2-{(3S)-3-[(tert-butoxycarbonyl)amino]pyrrolidin-1-yl}-3-(4,5-dihydroimidazo[1,5-a]quinolin-3-yl)propanoate

To a solution of ethyl 2-{(3S)-3-[(tert-butoxycarbonyl)amino]pyrrolidin-1-yl}-3-(4,5-dihydroimidazo[1,5-a]quinolin-3-yl)propanoate (96 mg) in methanol (3 ml), an aqueous solution of 2 M sodium hydroxide (2 ml) was added and the mixture was stirred for an hour at 60° C. The reaction mixture was evaporated under reduced pressure; after adding water, the aqueous layer was washed with ethyl acetate. The aqueous layer was neutralized with an aqueous solution of 1 M HCl and after adding sodium chloride, extraction was performed with chloroform and then with a mixed solvent of 5:1 chloroform/methanol. After drying over anhydrous magnesium sulfate, the desiccant was filtered off and the solvents were removed in vacuo. The resulting residue was dissolved in chloroform (2 ml) and after adding (1R)-1-phenylethanol (34 mg), 4-dimethylaminopyridine (4 mg) and N-[3-(dimethylamino)propyl]-N′-ethylcarbodiimide hydrochloride (52 mg), the mixture was stirred for 15 hours at room temperature. The reaction mixture was purified by silica gel column chromatography (eluent:ethyl acetate→chloroform/methanol=95:5), then by another run of silica gel column chromatography (eluent: ethyl acetate/2-propanol=95:5) to give the titled compound (21 mg) as a colorless gum of low-polarity compound.

MS (ESI/APCI Dual) m/z 531 [M+H]⁺

¹H NMR (300 MHz, CHLOROFORM-d) δ ppm 1.38 (d, J=6.5 Hz, 3H), 1.40 (s, 9H), 1.51-1.75 (m, 1H), 1.98-2.14 (m, 1H), 2.53-2.72 (m, 2H), 2.74-3.06 (m, 8H), 3.80 (t, J=7.7 Hz, 1H), 4.02-4.14 (m, 1H), 5.07-5.16 (m, 1H), 5.85 (q, J=6.5 Hz, 1H), 7.12-7.19 (m, 1H), 7.21-7.34 (m, 7H), 7.37-7.43 (m, 1H), 7.95 (s, 1H)

(9) Synthesis of (2S)-2-{(3S)-3-[(tert-butoxycarbonyl)amino]pyrrolidin-1-yl}-3-(4,5-dihydroimidazo[1,5-a]quinolin-3-yl)propanoic acid

To a solution of (1R)-1-phenylethyl (2S)-2-{(3S)-3-[(tert-butoxycarbonyl)amino]pyrrolidin-1-yl}-3-(4,5-dihydroimidazo[1,5-a]quinolin-3-yl)propanoate (231 mg) in methanol (20 ml), 10% palladium-activated carbon (100 mg) was added and the mixture was stirred for 9 hours at room temperature under hydrogen purge. The reaction mixture was filtered through Celite and the solvent was removed in vacuo. The resulting residue was purified by silica gel column chromatography (eluent: chloroform/methanol=9:1 to 7:3). To a solution of the recovered material (10 mg) in methanol (2 ml), 10% palladium-activated carbon (20 mg) was added and the mixture was stirred for 16 hours at room temperature under hydrogen purge. The reaction mixture was filtered through Celite and the solvent was removed in vacuo. The resulting residue was purified by silica gel column chromatography (eluent: chloroform/methanol=9:1 to 7:3). The products were combined to give the titled compound (195 mg) as a colorless powder.

MS (ESI/APCI Dual) m/z 427 [M+H]⁺

¹H NMR (600 MHz, CHLOROFORM-d) δ ppm 1.42 (s, 9H), 2.03-2.11 (m, 1H), 2.23-2.32 (m, 1H), 2.87-2.97 (m, 4H), 3.11-3.20 (m, 1H), 3.28-3.62 (m, 4H), 3.70-3.80 (m, 1H), 3.86-3.95 (m, 1H), 4.38-4.47 (m, 1H), 6.56-6.68 (m, 1H), 7.18-7.24 (m, 1H), 7.27-7.34 (m, 2H), 7.38-7.42 (m, 1H), 8.03-8.10 (m, 1H)

(10) Synthesis of (2S)-2-[(3S)-3-aminopyrrolidin-1-yl}-3-(4,5-dihydroimidazo[1,5-a]quinolin-3-yl)propanoic acid trihydrochloride

An aqueous solution of 6 M HCl (4 ml) having (2S)-2-{(3S)-[(tert-butoxycarbonyl)amino]pyrrolidin-1-yl}-3-(4,5-dihydroimidazo[1,5-a]quinolin-3-yl)propanoic acid (195 mg) dissolved therein was stirred for 2 hours at room temperature and, thereafter, the solvent was removed in vacuo to give the titled compound (compound 1; 180 mg) as a light brown amorphous mass.

MS (ESI/APCI Dual) m/z 327 [M+H]⁺

¹H NMR (600 MHz, DEUTERIUM OXIDE) δ ppm 2.26-2.36 (m, 1H), 2.70-2.77 (m, 1H), 2.96-3.07 (m, 4H), 3.36-3.43 (m, 1H), 3.51-3.58 (m, 1H), 3.66-3.73 (m, 1H), 3.77-3.83 (m, 1H), 3.86-3.92 (m, 1H), 3.96-4.02 (m, 1H), 4.08-4.17 (m, 1H), 4.26-4.34 (m, 1H), 7.42-7.51 (m, 3H), 7.70 (d, J=7.8 Hz, 1H), 9.29 (s, 1H)

[α]_{D}²⁵=+28.1 (c=0.25, H₂O)

Optical purity: >99% ee

r.t.: 15.30 min

### Example 2

### Synthesis of (2S)-2-[(3S)-3-aminopyrrolidin-1-yl]-3-(7-methyl-4,5-dihydroimidazo[1,5-a]quinolin-3-yl)propanoic acid trihydrochloride

(1) Synthesis of ethyl 7-methyl-4,5-dihydroimidazo[1,5-a]quinoline-3-carboxylate

Using 6-methyl-3,4-dihydroquinolin-2(1H)-one (3.3 g), reaction and purification were performed by repeating the procedures of (1) in Example 1 to give the titled compound (2.14 g) as a colorless powder.

MS (ESI/APCI Dual) m/z 279 [M+Na]⁺

¹H NMR (300 MHz, CHLOROFORM-d) δ ppm 1.42 (t, J=7.1 Hz, 3H), 2.37 (s, 3H), 2.87-2.94 (m, 2H), 3.29-3.37 (m, 2H), 4.40 (q, J=7.1 Hz, 2H), 7.11-7.16 (m, 2H), 7.33-7.37 (m, 1H), 7.98 (s, 1H)

(2) Synthesis of 7-methyl-4,5-dihydroimidazo[1,5-a]quinoline-3-carbaldehyde

A solution of ethyl 7-methyl-4,5-dihydroimidazo[1,5-a]quinoline-3-carboxylate (2.98 g) in tetrahydrofuran (35 ml) was cooled to −78° C. and after adding diisobutylaluminum hydride (59.0 ml as 1.01 M toluene solution) dropwise, the mixture was stirred for an hour at the same temperature. Methanol (10 ml) was added to quench the reaction; thereafter, an aqueous solution of 15% citric acid (25 ml) was added and the mixture was stirred for an hour at room temperature. After extracting with chloroform, the organic layer was washed with brine. After drying over anhydrous magnesium sulfate, the desiccant was filtered off and the solvents were removed in vacuo. The resulting residue was purified by silica gel column chromatography (eluent: chloroform/ethyl acetate=7:3 to 1:1) to give the titled compound (2.03 g) as a colorless powder.

MS (ESI/APCI Dual) m/z 213 [M+H]⁺

¹H NMR (300 MHz, CHLOROFORM-d) δ ppm 2.38 (s, 3H), 2.89-2.96 (m, 2H), 3.29-3.37 (m, 2H), 7.13-7.19 (m, 2H), 7.36 (d, J=8.7 Hz, 1H), 8.03 (s, 1H), 10.00 (s, 1H)

(3) Synthesis of ethyl 2-(acetoxy)-3-(7-methyl-4,5-dihydroimidazo[1,5-a]quinolin-3-yl)-2-propenoate

To a solution of ethyl (acetoxy)(diethoxyphosphoryl)acetate (3.91 g) in tetrahydrofuran (95 ml), lithium chloride (522 mg) was added, followed by dropwise addition of 1,1,3,3-tetramethylguanidine (1.42 g) at −78° C., and the mixture was stirred for 15 minutes at the same temperature. A solution of 7-methyl-4,5-dihydroimidazo[1,5-a]quinoline-3-carbaldehyde (2.01 g) in tetrahydrofuran was added dropwise and the mixture was stirred for 3 hours as the temperature was raised from −78° C. to room temperature. A saturated aqueous solution of ammonium chloride was added under cooling with ice to quench the reaction. After evaporating the solvent, brine was added to the residue and extracting with chloroform was performed. After drying the organic layer with anhydrous sodium sulfate, the desiccant was filtered off and the solvents were removed in vacuo. The resulting residue was purified by silica gel column chromatography (eluent: chloroform/ethyl acetate=1:1) to give the titled compound (4.47 g) as a pale yellow powder.

¹H NMR (300 MHz, CHLOROFORM-d) δ ppm 1.20-1.43 (m, 3H), 2.22, 2.25 (s, 3H), 2.35 (s, 3H), 2.78-3.06 (m, 4H), 4.15-4.37 (m, 2H), 6.68, 7.35 (s, 1H), 7.09-7.17 (m, 2H), 7.28-7.34 (m, 1H), 7.98-8.02 (m, 1H)

(4) Synthesis of ethyl 2-(acetoxy)-3-(7-methyl-4,5-dihydroimidazo[1,5-a]quinolin-3-yl)propanoate

To a solution of ethyl 2-(acetoxy)-3-(7-methyl-4,5-dihydroimidazo[1,5-a]quinolin-3-yl)-2-propenoate (4.47 g) in 2:1 ethanol/tetrahydrofuran (71 ml), 10% palladium-activated carbon (894 mg) was added and the mixture was stirred for 15 hours at room temperature under hydrogen purge. The reaction mixture was filtered through Celite and the solvents were removed in vacuo to give the titled compound (4.42 g) as an unpurified dark yellow oil.

¹H NMR (300 MHz, CHLOROFORM-d) δ ppm 1.17-1.43 (m, 3H), 2.09 (s, 3H), 2.36 (s, 3H), 2.88 (s, 4H), 3.04-3.26 (m, 2H), 4.18-4.41 (m, 2H), 5.21-5.33 (m, 1H), 7.07-7.20 (m, 2H), 7.30-7.36 (m, 1H), 8.03 (s, 1H)

(5) Synthesis of ethyl 2-hydroxy-3-(7-methyl-4,5-dihydrorimidazo[1,5-a]quinolin-3-yl)propanoate

To a solution of ethyl 2-(acetoxy)-3-(7-methyl-4,5-dihydrorimidazo[1,5-a]quinolin-3-yl)propanoate (4.42 g) in ethanol (47 ml), sodium ethoxide (3.22 g as 20% ethanol solution) was added and the mixture was stirred for 3 hours at room temperature. The reaction mixture was evaporated under reduced pressure and after adding a saturated aqueous solution of ammonium chloride and brine, extracting with chloroform was performed. After drying the organic layer with anhydrous sodium sulfate, the desiccant was filtered off and the solvents were removed in vacuo. The resulting residue was purified by silica gel column chromatography (eluent: n-hexane/ethyl acetate=1:1 to 1:4) to give the titled compound (1.80 g) as a pale yellow powder.

MS (ESI/APCI Dual) m/z 301 [M+H]⁺

¹H NMR (300 MHz, CHLOROFORM-d) δ ppm 1.26 (t, J=7.1 Hz, 3H), 2.35 (s, 3H), 2.85 (s, 4H), 2.92-3.15 (m, 2H), 4.07-4.31 (m, 2H), 4.46-4.62 (m, 1H), 7.04-7.15 (m, 2H), 7.27-7.34 (m, 1H), 7.91 (s, 1H)

(6) Synthesis of ethyl 2-{(3S)-3-[(tert-butoxycarbonyl)-amino]pyrrolidin-1-yl}-3-(7-methyl-4,5-dihydroimidazo[1,5-a]quinolin-3-yl)propanoate

Using ethyl 2-hydroxy-3-(7-methyl-4,5-dihydroimidazo[1,5-a]quinolin-3-yl)propanoate (1.80 g), reaction and purification were performed by repeating the procedures of (7) in Example 1 to give the titled compound (2.80 g) as a yellow oil.

MS (ESI/APCI Dual) m/z 469 [M+H]⁺

¹H NMR (300 MHz, CHLOROFORM-d) δ ppm 1.09-1.21 (m, 3H), 1.42, 1.43 (s, 9H), 1.55-1.72 (m, 1H), 2.07-2.27 (m, 1H), 2.34 (s, 3H), 2.52-3.10 (m, 10H), 3.62-3.76 (m, 1H), 3.98-4.24 (m, 3H), 4.92-5.28 (m, 1H), 7.04-7.14 (m, 2H), 7.24-7.32 (m, 1H), 7.911, 7.905 (s, 1H)

(7) Synthesis of 2-{(3S)-3-[(tert-butoxycarbonyl)-amino]pyrrolidin-1-yl}-3-(7-methyl-4,5-dihydroimidazo[1,5-a]quinolin-3-yl)propanoic acid

To a solution of ethyl 2-{(3S)-3-[(tert-butoxycarbonyl)-amino]pyrrolidin-1-yl}-3-(7-methyl-4,5-dihydroimidazo[1,5-a]quinolin-3-yl)propanoate (2.80 g) in methanol (30 ml), an aqueous solution of 2 M sodium hydroxide (14.9 ml) was added and the mixture was stirred for 2 hours at 60° C. The reaction mixture was evaporated under reduced pressure and after adding water, the aqueous layer was washed with diethyl ether. The aqueous layer was neutralized with an aqueous solution of 15% citric acid and extracting was performed with 5:1 chloroform/methanol. After drying over anhydrous sodium sulfate, the desiccant was filtered off and the solvents were removed in vacuo. The resulting residue was purified by silica gel column chromatography (eluent: chloroform/methanol=10:1 to 4:1) to give the titled compound (2.63 g) as a light brown powder.

MS (ESI/APCI Dual) m/z 441 [M+H]⁺

¹H NMR (300 MHz, CHLOROFORM-d) δ ppm 1.41 (s, 9H), 1.94-2.15 (m, 1H), 2.15-2.33 (m, 1H), 2.33 (s, 3H), 2.76-2.98 (m, 4H), 2.98-3.84 (m, 6H), 3.89 (t, J=6.2 Hz, 1 H), 4.33-4.53 (m, 1H), 6.81-6.98 (m, 1H), 7.03-7.15 (m, 2H), 7.21-7.34 (m, 1H), 7.96, 7.97 (s, 1H)

(8) Synthesis of (1R)-1-phenylethyl (2S)-2-{(3S)-3-[(tert-butoxycarbonyl)-amino]pyrrolidin-1-yl}-3-(7-methyl-4,5-dihydroimidazo[1,5-a]quinolin-3-yl)propanoate

To a solution of 2-{(3S)-3-[(tert-butoxycarbonyl)-amino]pyrrolidin-1-yl}-3-(7-methyl-4,5-dihydroimidazo[1,5-a]quinolin-3-yl)propanoic acid (2.23 g) in chloroform (25 ml), (1R)-1-phenylethanol (927 mg), 4-dimethylaminopyridine (62 mg) and N-[3-(dimethylamino)propyl]-N′-ethylcarbodiimide hydrochloride (1.46 g) were added and the mixture was stirred for 15 hours at room temperature. After adding a saturated solution of sodium hydrogencarbonate, extracting with chloroform was performed. After drying over anhydrous sodium sulfate, the desiccant was filtered off and the solvent was removed in vacuo. The resulting residue was purified by silica gel column chromatography (eluent: n-hexane/ethyl acetate=1:1 to 1:4) to give the titled compound (660 mg) as a brown oil.

MS (ESI/APCI Dual) m/z 545 [M+H]⁺

¹H NMR (300 MHz, CHLOROFORM-d) δ ppm 1.32-1.49 (m, 12H), 1.51-1.72 (m, 1H), 1.98-2.16 (m, 1H), 2.34 (s, 3H), 2.50-3.07 (m, 10H), 3.79 (t, J=7.7 Hz, 1H), 4.01-4.15 (m, 1H), 5.07-5.20 (m, 1H), 5.84 (q, J=6.5 Hz, 1H), 7.03-7.14 (m, 2H), 7.22-7.36 (m, 6H), 7.92 (s, 1H)

(9) Synthesis of (2S)-2-{(3S)-3-[(tert-butoxycarbonyl)-amino]pyrrolidin-1-yl}-3-(7-methyl-4,5-dihydroimidazo[1,5-a]quinolin-3-yl)propanoic acid

To a solution of (1R)-1-phenylethyl (2S)-2-{(3S)-3-[(tert-butoxycarbonyl)-amino]pyrrolidin-1-yl}-3-(7-methyl-4,5-dihydroimidazo[1,5-a]quinolin-3-yl)propanoate (660 mg) in methanol (12 ml), 10% palladium-activated carbon (132 mg) was added and the mixture was stirred for 5 hours at 60° C. under hydrogen purge. The reaction mixture was filtered through Celite and the solvent was removed in vacuo. The resulting residue was purified by silica gel column chromatography (eluent: chloroform/methanol=10:1) to give the titled compound (280 mg) as a light brown powder.

MS (ESI/APCI Dual) m/z 441 [M+H]⁺

¹H NMR (300 MHz, CHLOROFORM-d) δ ppm 1.42 (s, 9H), 2.08 (br. s, 1H), 2.18-2.33 (m, 1H), 2.34 (s, 3H), 2.75-2.99 (m, 4H), 3.01-3.19 (m, 1H), 3.21-3.59 (m, 3H), 3.69-3.85 (m, 1H), 3.89 (t, J=6.1 Hz, 1H), 4.30-4.54 (m, 1H), 6.77-6.93 (m, 1H), 7.03-7.17 (m, 1H), 7.23-7.33 (m, 2H), 7.97 (s, 1H)

(10) Synthesis of (2S)-2-[(3S)-3-aminopyrrolidin-1-yl]-3-(7-methyl-4,5-dihydroimidazo[1,5-a]quinolin-3-yl)propanoic acid trihydrochloride

To a suspension of (2S)-2-{(3S)-3-[(tert-butoxycarbonyl)-amino]pyrrolidin-1-yl}-3-(7-methyl-4,5-dihydroimidazo[1,5-a]quinolin-3-yl)propanoic acid (140 mg) in ethyl acetate (1.59 ml), a 4 M HCl solution in ethyl acetate (1.59 ml) was added and the mixture was stirred for 4 hours at room temperature; thereafter, the solvent was removed in vacuo to give the titled compound (compound 2; 110 mg) as a brown powder.

MS (ESI/APCI Dual) m/z 341 [M+H]⁺

¹H NMR (600 MHz, DEUTERIUM OXIDE) δ ppm 2.23-2.33 (m, 1H), 2.37 (s, 3H), 2.67-2.77 (m, 1H), 2.96 (s, 4H), 3.30-3.38 (m, 1H), 3.44-3.52 (m, 1H), 3.61-3.69 (m, 1H), 3.73-3.80 (m, 1H), 3.82-3.90 (m, 1H), 3.90-3.96 (m, 1H), 3.96-4.02 (m, 1H), 4.22-4.33 (m, 1H), 7.26-7.35 (m, 2H), 7.55-7.63 (m, 1H), 9.21 (s, 1H)

### Example 3

### Synthesis of (2S)-3-(4,5-dihydroimidazo[1,5-a]quinolin-3-yl)-2-{(3S)-3-[({1-[(2-methylpropanoyl)oxy]ethoxy}carbonyl)amino]pyrrolidin-1-yl}propanoic acid

(1) Synthesis of (2S)-2-[(3S)-3-aminopyrrolidin-1-yl]-(3-(4,5-dihydroimidazo[1,5-a]quinolin-3-yl)propanoic acid

To a solution of (2S)-2-{(3S)-3-[(tert-butoxycarbonyl)amino]pyrrolidin-1-yl}-(3-(4,5-dihydroimidazo[1,5-a]quinolin-3-yl)propanoic acid (400 mg) in ethyl acetate (10 ml), a 4 M HCl solution in ethyl acetate (10 ml) was added and the mixture was stirred for 4 hours at room temperature. The solvent was removed in vacuo and after adding water and Amberlite IRA-67 (3.0 g) to the resulting residue, the mixture was stirred for 30 minutes at room temperature. The reaction mixture was filtered and the filtrate was concentrated under reduced pressure to give the titled compound (288 mg) as a light brown powder.

MS (ESI) m/z 327 [M+H]⁺

¹H NMR (600 MHz, DEUTERIUM OXIDE) δ ppm 1.84-1.92 (m, 1H), 2.30-2.37 (m, 1H), 2.82-3.04 (m, 8H), 3.16-3.28 (m, 2H), 3.37-3.45 (m, 1H), 3.80-3.88 (m, 1H), 7.24-7.29 (m, 1H), 7.35-7.43 (m, 2H), 7.55-7.60 (m, 1H), 8.17 (s, 1H)

(2) Synthesis of (2S)-(3-(4,5-dihydroimidazo[1,5-a]quinolin-3-yl)-2-{(3S)-3-[({1-[(2-methylpropanoyl)oxy]ethoxy}carbonyl)amino]pyrrolidin-1-yl}propanoic acid

To a solution of (2S)-2-[(3S)-3-aminopyrrolidin-1-yl]-(3-(4,5-dihydroimidazo[1,5-a]quinolin-3-yl)propanoic acid (140 mg) in N,N-dimethylformamide (5 ml), 1-{[(4-nitrophenoxy)carbonyl]oxy}ethyl 2-methyl propanoate (155 mg) was added and the mixture was stirred overnight at room temperature. Water was added to the reaction mixture, which was then washed with diethyl ether. The aqueous layer was extracted with chloroform and the organic layer was dried over anhydrous sodium sulfate, followed by removing the solvents in vacuo. The resulting residue was purified by diol silica gel column chromatography (eluent: chloroform/methanol=100:0 to 80:20) and after adding diethyl ether, the recovered fractions were reduced to a powder form, which was subjected to decantation to give the titled compound (compound 3; 77 mg) as a yellow powder.

MS (ESI/APCI Dual) m/z 485 [M+H]⁺

¹H NMR (600 MHz, DMSO-d₆) δ ppm 1.04-1.09 (m, 6H), 1.35-1.41 (m, 3H), 1.57-1.65 (m, 1H), 1.97-2.06 (m, 1H), 2.57-2.63 (m, 1H), 2.71-2.92 (m, 9H), 3.02-3.07 (m, 1H), 3.46-3.52 (m, 1H), 3.88-3.97 (m, 1H), 6.61-6.66 (m, 1H), 7.15-7.19 (m, 1H), 7.29-7.38 (m, 2H), 7.63-7.68 (m, 1H), 7.68-7.72 (m, 1H), 8.27 (s, 1H)

### Example 4

### Synthesis of (2S)-3-(4,5-dihydroimidazo[1,5-a]quinolin-3-yl)-2-[(3S)-3-({[5-methyl-2-oxo-1,3-dioxol-4-yl)methoxy]carbonyl}amino)pyrrolidin-1-yl]propanoic acid

To a solution of (2S)-2-[(3S)-3-aminopyrrolidin-1-yl]-(3-(4,5-dihydroimidazo[1,5-a]quinolin-3-yl)propanoic acid (148 mg) in N,N-dimethylformamide (5 ml), (5-methyl-2-oxo-1,3-dioxol-4-yl)methyl 4-nitrophenyl carbonate (159 mg) was added and the mixture was stirred overnight at room temperature. Water was added to the reaction mixture, which was then washed with diethyl ether. The aqueous layer was extracted with chloroform and the organic layer was dried over anhydrous sodium sulfate, followed by removing the solvents in vacuo. The resulting residue was purified by diol silica gel column chromatography (eluent: chloroform/methanol=100:0 to 90:10) and after adding diethyl ether, the fractions were reduced to a powder form, which was subjected to decantation. After adding water, azeotropic distillation was performed twice to give the titled compound (compound 4; 55 mg) as a light brown amorphous mass.

MS (ESI/APCI Dual) m/z 483 [M+H]⁺

¹H NMR (600 MHz, DMSO-d₆) δ ppm 1.56-1.66 (m, 1H), 1.98-2.07 (m, 1H), 2.15 (s, 3H), 2.57-2.64 (m, 1H), 2.72-2.93 (m, 8H), 3.02-3.10 (m, 1H), 3.46-3.53 (m, 1H), 3.91-4.01 (m, 1H), 4.79-4.91 (m, 2H), 7.15-7.20 (m, 1H), 7.30-7.38 (m, 2H), 7.55-7.60 (m, 1H), 7.68-7.72 (m, 1H), 8.27 (s, 1H)

### Example 5

### Synthesis of ethyl (2S)-2-[(3S)-3-aminopyrrolidin-1-yl]-3-(4,5-dihydroimidazo[1,5-a]quinolin-3-yl)propanoate trihydrochloride

(1) Synthesis of ethyl (2S)-2-{(3S)-3-[(tert-butoxycarbonyl)amino]pyrrolidin-1-yl}-(3-(4,5-dihydroimidazo[1,5-a]quinolin-3-yl)propanoate

To a solution of (2S)-2-{(3S)-3-[(tert-butoxycarbonyl)amino]pyrrolidin-1-yl}-(3-(4,5-dihydroimidazo[1,5-a]quinolin-3-yl)propanoic acid (154 mg) in N,N-dimethylformamide (2 ml), cesium carbonate (178 mg) and ethyl iodide (45 μl) were added under cooling with ice and the mixture was stirred for an hour at the same temperature. Water was added to the reaction mixture, followed by extracting with chloroform. Brine was added to the aqueous layer, followed by extracting with chloroform. After drying the combined organic layers with anhydrous magnesium sulfate, the desiccant was filtered off and the solvents were removed in vacuo. The resulting residue was purified by silica gel column chromatography (eluent: n-hexane/ethyl acetate=1:4 to 0:1→chloroform/methanol=9:1). Since the recovered fractions contained N,N-dimethylformamide, they were dissolved in ethyl acetate and washed with brine three times. After drying the organic layer with anhydrous magnesium sulfate, the desiccant was filtered off and the solvents were removed in vacuo. The resulting residue was purified by silica gel column chromatography (eluent: n-hexane/ethyl acetate=1:4 to 0:1→chloroform/methanol=19:1) to give the titled compound (61 mg) as a colorless oil.

MS (ESI/APCI Dual) m/z 455 [M+H]⁺

¹H NMR (300 MHz, CHLOROFORM-d) δ ppm 1.18 (t, J=7.1 Hz, 3H), 1.41 (s, 9H), 1.61-1.72 (m, 1H), 2.08-2.24 (m, 1H), 2.58-2.68 (m, 1H), 2.71-2.79 (m, 1H), 2.82-3.06 (m, 8H), 3.67-3.75 (m, 1H), 4.10 (q, J=7.1 Hz, 2H), 4.07-4.20 (m, 1H), 5.15-5.26 (m, 1H), 7.13-7.19 (m, 1H), 7.25-7.33 (m, 2H), 7.38-7.41 (m, 1H), 7.95 (s, 1H)

(2) Synthesis of ethyl (2S)-2-[(3S)-3-aminopyrrolidin-1-yl]-3-(4,5-dihydroimidazo[1,5-a]quinolin-3-yl)propanoate trihydrochloride

An aqueous solution of 4 M HCl (3 ml) having ethyl (2S)-2-{(3S)-3-[(tert-butoxycarbonyl)amino]pyrrolidin-1-yl}-(3-(4,5-dihydroimidazo[1,5-a]quinolin-3-yl)propanoate (59 mg) dissolved therein was stirred for 6 hours at room temperature. The solvent was removed in vacuo to give the titled compound (compound 5; 58 mg) as a light brown amorphous mass.

MS (ESI/APCI Dual) m/z 355 [M+H]⁺

¹H NMR (600 MHz, DEUTERIUM OXIDE) δ ppm 1.11 (t, J=7.1 Hz, 3H), 2.11-2.21 (m, 1H), 2.56-2.64 (m, 1H), 2.92-3.09 (m, 4H), 3.32-3.45 (m, 3H), 3.50-3.60 (m, 2H), 3.68-3.74 (m, 1H), 4.12-4.18 (m, 1H), 4.20 (q, J=7.1 Hz, 2H), 4.24-4.30 (m, 1H), 7.44-7.52 (m, 3H), 7.70-7.74 (m, 1H), 9.33 (s, 1H)

### Example 6

### Synthesis of butyl (2S)-2-[(3S)-3-aminopyrrolidin-1-yl]-3-(4,5-dihydroimidazo[1,5-a]quinolin-3-yl)propanoate trihydrochloride

(1) Synthesis of butyl (2S)-2-{(3S)-3-[(tert-butoxycarbonyl)amino]pyrrolidin-1-yl}-(3-(4,5-dihydroimidazo[1,5-a]quinolin-3-yl)propanoate

To a solution of (2S)-2-{(3S)-3-[(tert-butoxycarbonyl)amino]pyrrolidin-1-yl}-(3-(4,5-dihydroimidazo[1,5-a]quinolin-3-yl)propanoic acid (150 mg) in N,N-dimethylformamide (5 ml), cesium carbonate (173 mg) and 1-iodobutane (129 mg) were added under cooling with ice and the mixture was stirred for two hours at room temperature. Water was added to the reaction mixture under cooling with ice, followed by extracting with ethyl acetate. After washing the organic layer with water and brine, drying was performed with anhydrous sodium sulfate; thereafter, the desiccant was filtered off and the solvents were removed in vacuo. The resulting residue was purified by silica gel column chromatography (eluent: chloroform/methanol=97:3 to 90:10) and then by NH silica gel column chromatography (eluent: n-hexane/ethyl acetate=100:0 to 80:20) to give the titled compound (130 mg) as a yellow gum.

MS (ESI/APCI Dual) m/z 483 [M+H]⁺

¹H NMR (300 MHz, CHLOROFORM-d) δ ppm 0.79-0.86 (m, 3H), 1.16-1.32 (m, 2H), 1.41 (s, 9H), 1.45-1.58 (m, 2H), 2.09-2.26 (m, 1H), 2.55-2.70 (m, 1H), 2.72-2.80 (m, 1H), 2.81-3.07 (m, 9H), 3.68-3.81 (m, 1H), 3.98-4.08 (m, 2H), 4.10-4.22 (m, 1H), 5.15-5.29 (m, 1H), 7.12-7.20 (m, 1H), 7.26-7.34 (m, 2H), 7.37-7.43 (m, 1H), 7.95 (s, 1H)

(2) Synthesis of butyl (2S)-2-[(3S)-3-aminopyrrolidin-1-yl]-3-(4,5-dihydroimidazo[1,5-a]quinolin-3-yl)propanoate trihydrochloride

To a solution of butyl (2S)-2-{(3S)-3-[(tert-butoxycarbonyl)amino]pyrrolidin-1-yl}-(3-(4,5-dihydroimidazo[1,5-a]quinolin-3-yl)propanoate (130 mg) in ethyl acetate (4 ml), a 4 M HCl solution in ethyl acetate (4 ml) was added and the mixture was stirred for 3 hours at room temperature. The solvent was removed in vacuo and water was added to the resulting residue, which was subjected to azeotropic distillation to give the titled compound (compound 6; 112 mg) as a light brown amorphous mass.

MS (ESI/APCI Dual) m/z 383 [M+H]⁺

¹H NMR (600 MHz, DEUTERIUM OXIDE) δ ppm 0.67 (t, J=7.3 Hz, 3H), 1.01-1.12 (m, 2H), 1.33-1.49 (m, 2H), 1.98-2.06 m, 1H), 2.44-2.52 (m, 1H), 2.91-3.15 (m, 6H), 3.25-3.44 (m, 4H), 3.93-3.98 (m, 1H), 3.99-4.15 (m, 3H), 7.43-7.52 (m, 3H), 7.69-7.74 (m, 1H), 9.33 (s, 1H)

### Example 7

### Synthesis of heptyl (2S)-2-[(3S)-3-aminopyrrolidin-1-yl]-3-(4,5-dihydroimidazo[1,5-a]quinolin-3-yl)propanoate trihydrochloride

(1) Synthesis of heptyl (2S)-2-{(3S)-3-[(tert-butoxycarbonyl)amino]pyrrolidin-1-yl}-(3-(4,5-dihydroimidazo[1,5-a]quinolin-3-yl)propanoate

To a solution of (2S)-2-{(3S)-3-[(tert-butoxycarbonyl)amino]pyrrolidin-1-yl}-(3-(4,5-dihydroimidazo[1,5-a]quinolin-3-yl)propanoic acid (208 mg) in N,N-dimethylformamide (5 ml), cesium carbonate (241 mg) and 1-iodoheptane (167 mg) were added under cooling with ice and the mixture was stirred for 1.5 hours at the same temperature and for an additional two hours at room temperature. Water was added to the reaction mixture under cooling with ice, followed by extracting with ethyl acetate. After washing the organic layer with water twice and with brine, drying was performed with anhydrous sodium sulfate; thereafter, the desiccant was filtered off and the solvents were removed in vacuo. The resulting residue was purified by silica gel column chromatography (eluent: chloroform/methanol=100:0 to 90:10) and then by NH silica gel column chromatography (eluent: n-hexane/ethyl acetate=100:0 to 80:20) to give the titled compound (233 mg) as a colorless amorphous mass.

MS (ESI/APCI Dual) m/z 525 [M+H]⁺

¹H NMR (300 MHz, CHLOROFORM-d) δ ppm 0.79-0.88 (m, 3H), 1.10-1.28 (m, 8H), 1.41 (s, 9H), 1.36-1.70 (m, 3H), 2.07-2.23 (m, 1H), 2.56-2.68 (m, 1H), 2.70-2.79 (m, 1H), 2.81-3.07 (m, 7H), 3.67-3.76 (m, 1H), 3.97-4.05 (m, 2H), 4.08-4.22 (m, 1H), 5.15-5.27 (m, 1H), 7.12-7.19 (m, 1H), 7.24-7.33 (m, 2H), 7.37-7.42 (m, 1H), 7.94 (s, 1H)

(2) Synthesis of heptyl (2S)-2-[(3S)-3-aminopyrrolidin-1-yl]-3-(4,5-dihydroimidazo[1,5-a]quinolin-3-yl)propanoate trihydrochloride

To a solution of heptyl (2S)-2-{(3S)-3-[(tert-butoxycarbonyl)amino]pyrrolidin-1-yl}-(3-(4,5-dihydroimidazo[1,5-a]quinolin-3-yl)propanoate (233 mg) in ethyl acetate (5 ml), a 4 M HCl solution in ethyl acetate (5 ml) was added and the mixture was stirred for two hours at room temperature. The solvent was removed in vacuo and water was added to the resulting residue, which was subjected to azeotropic distillation to give the titled compound (compound 7; 195 mg) as a light brown amorphous mass.

MS (ESI/APCI Dual) m/z 425 [M+H]⁺

¹H NMR (600 MHz, DEUTERIUM OXIDE) δ ppm 0.71 (t, J=7.3 Hz, 3H), 0.90-1.08 (m, 8H), 1.32-1.44 (m, 2H), 1.90-1.98 (m, 1H), 2.36-2.44 (m, 1H), 2.79-3.31 (m, 10H), 3.70-3.75 (m, 1H), 3.92-4.02 (m, 2H), 4.09-4.15 (m, 1H), 7.41-7.51 (m, 3H), 7.68-7.72 (m, 1H), 9.20 (s, 1H)

### Example 8

### Synthesis of propan-2-yl (2S)-2-[(3S)-3-aminopyrrolidin-1-yl]-3-(4,5-dihydroimidazo[1,5-a]quinolin-3-yl)propanoate trihydrochloride

(1) Synthesis of propan-2-yl (2S)-2-{(3S)-3-[(tert-butoxycarbonyl)amino]pyrrolidin-1-yl}-(3-(4,5-dihydroimidazo[1,5-a]quinolin-3-yl)propanoate

To a solution of (2S)-2-{(3S)-3-[(tert-butoxycarbonyl)amino]pyrrolidin-1-yl}-(3-(4,5-dihydroimidazo[1,5-a]quinolin-3-yl)propanoic acid (300 mg) in N,N-dimethylformamide (7 ml), cesium carbonate (342 mg) and 2-iodopropane (178 mg) were added under cooling with ice and the mixture was stirred for an hour at the same temperature and for an additional six hours at room temperature. Water was added to the reaction mixture under cooling with ice, followed by extracting with ethyl acetate. After washing the organic layer with water twice and with brine, drying was performed with anhydrous sodium sulfate; thereafter, the desiccant was filtered off and the solvents were removed in vacuo. The resulting residue was purified by silica gel column chromatography (eluent: chloroform/methanol=100:0 to 90:10) to give the titled compound (290 mg) as a light brown gum.

MS (ESI/APCI Dual) m/z 469 [M+H]⁺

¹H NMR (300 MHz, CHLOROFORM-d) δ ppm 1.08 (d, J=6.2 Hz, 3H), 1.21 (d, J=6.2 Hz, 3H), 1.41 (s, 9H), 1.62-1.72 (m, 1H), 2.07-2.25 (m, 1H), 2.57-2.70 (m, 1H), 2.71-2.81 (m, 1H), 2.82-3.05 (m, 8H), 3.64-3.75 (m, 1H), 4.07-4.22 (m, 1H), 4.90-5.03 (m, 1H), 5.16-5.30 (m, 1H), 7.11-7.20 (m, 1H), 7.26-7.34 (m, 2H), 7.36-7.43 (m, 1H), 7.95 (s, 1H)

(2) Synthesis of propan-2-yl (2S)-2-[(3S)-3-aminopyrrolidin-1-yl]-3-(4,5-dihydroimidazo[1,5-a]quinolin-3-yl)propanoate trihydrochloride

To a solution of propan-2-yl (2S)-2-{(3S)-3-[(tert-butoxycarbonyl)amino]pyrrolidin-1-yl}-(3-(4,5-dihydroimidazo[1,5-a]quinolin-3-yl)propanoate (290 mg) in ethyl acetate (5 ml), a 4 M HCl solution in ethyl acetate (5 ml) was added and the mixture was stirred for two hours at room temperature. The solvent was removed in vacuo and water was added to the resulting residue, which was subjected to azeotropic distillation to give the titled compound (compound 8; 261 mg) as a brown amorphous mass.

MS (ESI/APCI Dual) m/z 369 [M+H]⁺

¹H NMR (600 MHz, DEUTERIUM OXIDE) δ ppm 1.01 (d, J=6.4 Hz, 3H), 1.19 (d, J=6.4 Hz, 3H), 1.99-2.07 (m, 1H), 2.45-2.52 (m, 1H), 2.93-3.08 (m, 5H), 3.10-3.14 (m, 1H), 3.25-3.33 (m, 2H), 3.36-3.44 (m, 2H), 3.91-3.96 (m, 1H), 4.00-4.06 (m, 1H), 4.92-4.99 (m, 1H), 7.43-7.51 (m, 3H), 7.70-7.73 (m, 1H), 9.30 (s, 1H)

### Example 9

### Synthesis of 2-methylpropyl (2S)-2-[(3S)-3-aminopyrrolidin-1-yl]-3-(4,5-dihydroimidazo[1,5-a]quinolin-3-yl)propanoate trihydrochloride

(1) Synthesis of 2-methylpropyl (2S)-2-{(3S)-3-[(tert-butoxycarbonyl)amino]pyrrolidin-1-yl}-(3-(4,5-dihydroimidazo[1,5-a]quinolin-3-yl)propanoate

To a solution of (2S)-2-{(3S)-3-[(tert-butoxycarbonyl)amino]pyrrolidin-1-yl}-(3-(4,5-dihydroimidazo[1,5-a]quinolin-3-yl)propanoic acid (150 mg) in N,N-dimethylformamide (5 ml), cesium carbonate (173 mg) and 1-iodo-2-methylpropane (129 mg) were added under cooling with ice and the mixture was stirred for 6 hours at room temperature. Water was added to the reaction mixture under cooling with ice, followed by extracting with ethyl acetate. After washing the organic layer with water and brine, drying was performed with anhydrous sodium sulfate; thereafter, the desiccant was filtered off and the solvents were removed in vacuo. The resulting residue was purified by silica gel column chromatography (eluent: chloroform/methanol=97:3 to 90:10) and then by NH silica gel column chromatography (eluent: n-hexane/ethyl acetate=100:0 to 80:20) to give the titled compound (122 mg) as a pale yellow brown gum.

MS (ESI/APCI Dual) m/z 483 [M+H]⁺

¹H NMR (300 MHz, CHLOROFORM-d) δ ppm 0.81 (d, J=3.6 Hz, 3H), 0.83 (d, J=3.6 Hz, 3H), 1.41 (s, 9H), 1.73-1.92 (m, 1H), 2.07-2.24 (m, 1H), 2.56-2.69 (m, 1H), 2.72-2.80 (m, 1H), 2.81-3.08 (m, 9H), 3.72-3.78 (m, 1H), 3.81 (d, J=6.7 Hz, 2H), 4.08-4.22 (m, 1H), 5.16-5.28 (m, 1H), 7.12-7.20 (m, 1H), 7.25-7.33 (m, 2H), 7.36-7.42 (m, 1H), 7.94 (s, 1H)

(2) Synthesis of 2-methylpropyl (2S)-2-[(3S)-3-aminopyrrolidin-1-yl]-3-(4,5-dihydroimidazo[1,5-a]quinolin-3-yl)propanoate trihydrochloride

To a solution of 2-methylpropyl (2S)-2-{(3S)-3-[(tert-butoxycarbonyl)amino]pyrrolidin-1-yl}-(3-(4,5-dihydroimidazo[1,5-a]quinolin-3-yl)propanoate (122 mg) in ethyl acetate (3 ml), a 4 M HCl solution in ethyl acetate (3 ml) was added and the mixture was stirred for 4 hours at room temperature. The solvent was removed in vacuo and water was added to the resulting residue, which was subjected to azeotropic distillation to give the titled compound (compound 9; 121 mg) as a light brown amorphous mass.

MS (ESI/APCI Dual) m/z 383 [M+H]⁺

¹H NMR (600 MHz, DEUTERIUM OXIDE) δ ppm 0.70 (d, J=2.8 Hz, 3H), 0.71 (d, J=2.8 Hz, 3H), 1.68-1.77 (m, 1H), 1.99-2.08 (m, 1H), 2.45-2.53 (m, 1H), 2.90-3.10 (m, 5H), 3.11-3.17 (m, 1H), 3.27-3.36 (m, 2H), 3.38-3.46 (m, 2H), 3.83-3.92 (m, 2H), 3.98-4.07 (m, 2H), 7.43-7.53 (m, 3H), 7.68-7.73 (m, 1H), 9.32 (s, 1H)

### Example 10

### Synthesis of cyclohexyl (2S)-2-[(3S)-3-aminopyrrolidin-1-yl]-3-(4,5-dihydroimidazo[1,5-a]quinolin-3-yl)propanoate trihydrochloride

(1) Synthesis of cyclohexyl (2S)-2-{(3S)-3-[(tert-butoxycarbonyl)amino]pyrrolidin-1-yl}-(3-(4,5-dihydroimidazo[1,5-a]quinolin-3-yl)propanoate

To a solution of (2S)-2-{(3S)-3-[(tert-butoxycarbonyl)amino]pyrrolidin-1-yl}-3-(4,5-dihydroimidazo[1,5-a]quinolin-3-yl)propanoic acid (150 mg) in N,N-dimethylformamide (5 ml), cesium carbonate (173 mg) and iodocyclohexane (147 mg) were added under cooling with ice and the mixture was stirred overnight at room temperature. Iodocyclohexane (294 mg) was further added and the mixture was stirred for 2 days at room temperature. Water was added to the reaction mixture, followed by extracting with ethyl acetate. After washing the organic layer with water and brine, drying was performed with anhydrous sodium sulfate; thereafter, the desiccant was filtered off and the solvents were removed in vacuo. The resulting residue was purified by silica gel column chromatography (eluent: chloroform/methanol=97:3 to 90:10) and then by NH silica gel column chromatography (eluent: n-hexane/ethyl acetate=100:0 to 80:20) to give the titled compound (43 mg) as a pale yellow amorphous mass.

MS (ESI/APCI Dual) m/z 509 [M+H]⁺

¹H NMR (300 MHz, CHLOROFORM-d) δ ppm 1.18-1.53 (m, 7H), 1.41 (s, 9H), 1.53-1.86 (m, 4H), 2.07-2.24 (m, 1H), 2.60-2.71 (m, 1H), 2.72-3.07 (m, 9H), 3.67-3.77 (m, 1H), 4.07-4.22 (m, 1H), 4.69-4.82 (m, 1H), 5.17-5.30 (m, 1H), 7.12-7.19 (m, 1H), 7.26-7.33 (m, 2H), 7.36-7.42 (m, 1H), 7.95 (s, 1H)

(2) Synthesis of cyclohexyl (2S)-2-[(3S)-3-aminopyrrolidin-1-yl]-3-(4,5-dihydroimidazo[1,5-a]quinolin-3-yl)propanoate trihydrochloride

To a solution of cyclohexyl (2S)-2-{(3S)-3-[(tert-butoxycarbonyl)amino]pyrrolidin-1-yl}-(3-(4,5-dihydroimidazo[1,5-a]quinolin-3-yl)propanoate (43 mg) in ethyl acetate (2 ml), a 4 M HCl solution in ethyl acetate (2 ml) was added and the mixture was stirred for 4 hours at room temperature. The solvent was removed in vacuo and water was added to the resulting residue, which was subjected to azeotropic distillation to give the titled compound (compound 10; 39 mg) as a light brown amorphous mass.

MS (ESI/APCI Dual) m/z 409 [M+H]⁺

¹H NMR (600 MHz, DEUTERIUM OXIDE) δ ppm 1.05-1.61 (m, 10H), 1.70-1.78 (m, 1H), 2.08-2.16 (m, 1H), 2.53-2.62 (m, 1H), 2.89-3.08 (m, 4H), 3.22-3.28 (m, 1H), 3.29-3.38 (m, 2H), 3.45-3.54 (m, 2H), 3.58-3.67 (m, 1H), 4.07-4.20 (m, 2H), 7.43-7.53 (m, 3H), 7.69-7.75 (m, 1H), 9.35 (s, 1H)

### Example 11

### Synthesis of 1-{[(cyclohexyloxy)carbonyl]oxy}ethyl (2S)-2-[(3S)-3-aminopyrrolidin-1-yl]-3-(4,5-dihydroimidazo[1,5-a]quinolin-3-yl)propanoate trihydrochloride

(1) Synthesis of 1-{[(cyclohexyloxy)carbonyl]oxy}ethyl (2S)-2-{(3S)-3-[(tert-butoxycarbonyl)amino]pyrrolidin-1-yl}-(3-(4,5-dihydroimidazo[1,5-a]quinolin-3-yl)propanoate

To a solution of (2S)-2-{(3S)-3-[(tert-butoxycarbonyl)amino]pyrrolidin-1-yl}-(3-(4,5-dihydroimidazo[1,5-a]quinolin-3-yl)propanoic acid (200 mg) in N,N-dimethylformamide (5 ml), cesium carbonate (231 mg) and cyclohexyl 1-iodoethyl carbonate (222 mg) were added under cooling with ice and the mixture was stirred for an hour at the same temperature and for an additional hour at room temperature. Water was added to the reaction mixture, followed by extracting with ethyl acetate. After washing the organic layer with water twice and with brine, drying was performed with anhydrous sodium sulfate; thereafter, the desiccant was filtered off and the solvents were removed in vacuo. The resulting residue was purified by silica gel column chromatography (eluent: chloroform/methanol=100:0 to 90:10) and then by NH silica gel column chromatography (eluent: n-hexane/ethyl acetate=100:0 to 80:20) to give the titled compound (81 mg) as a colorless gum.

MS (ESI/APCI Dual) m/z 597 [M+H]⁺

¹H NMR (300 MHz, CHLOROFORM-d) δ ppm 1.16-1.95 (m, 24H), 2.06-2.21 (m, 1H), 2.60-3.08 (m, 10H), 3.74-3.87 (m, 1H), 4.06-4.20 (m, 1H), 4.39-4.66 (m, 1H), 5.20-5.34 (m, 1H), 6.66-6.76 (m, 1H), 7.11-7.20 (m, 1H), 7.25-7.33 (m, 2H), 7.35-7.42 (m, 1H), 7.93, 7.94 (s, 1 H)

(1) Synthesis of 1-{[(cyclohexyloxy)carbonyl]oxy}ethyl (2S)-2-[(3S)-3-aminopyrrolidin-1-yl]-3-(4,5-dihydroimidazo[1,5-a]quinolin-3-yl)propanoate trihydrochloride

To a solution of 1-{[(cyclohexyloxy)carbonyl]oxy}ethyl (2S)-2-{(3S)-3-[(tert-butoxycarbonyl)amino]pyrrolidin-1-yl}-(3-(4,5-dihydroimidazo[1,5-a]quinolin-3-yl)propanoate (80 mg) in ethyl acetate (1 ml), a 4 M HCl solution in ethyl acetate (1 ml) was added and the mixture was stirred for two hours at room temperature. The solvent was removed in vacuo and water was added to the resulting residue, which was subjected to azeotropic distillation to give the titled compound (compound 11; 77 mg) as a brown amorphous mass.

MS (ESI/APCI Dual) m/z 497 [M+H]⁺

¹H NMR (600 MHz, DEUTERIUM OXIDE) δ ppm 1.01-1.25 (m, 5H), 1.37-1.72 (m, 8H), 1.89-1.98 (m, 1H), 2.35-2.44 (m, 1H), 2.76-3.30 (m, 10H), 3.77-3.82 (m, 1H), 3.90-3.97 (m, 1H), 4.13-4.21 (m, 0.5H), 4.38-4.47 (m, 0.5H), 6.56-6.61 (m, 0.5H), 6.64-6.69 (m, 0.5H), 7.3.7-7.52 (m, 3H), 7.69-7.74 (m, 1H), 9.12, 9.15 (s, 1H)

### Example 12

### Synthesis of 1-{[(cyclohexyloxy)carbonyl]oxy}-2-methylpropyl (2S)-2-[(3S)-3-aminopyrrolidin-1-yl]-3-(4,5-dihydroimidazo[1,5-a]quinolin-3-yl)propanoate trihydrochloride

(1) Synthesis of 1-{([(cyclohexyloxy)carbonyl]oxy}-2-methylpropyl (2S)-2-{(3S)-3-[(tert-butoxycarbonyl)amino]pyrrolidin-1-yl}-(3-(4,5-dihydroimidazo[1,5-a]quinolin-3-yl)propanoate

To a solution of (2S)-2-[(3S)-3-[(tert-butoxycarbonyl)amino]pyrrolidin-1-yl]-(3-(4,5-dihydroimidazo[1,5-a]quinolin-3-yl)propanoic acid (150 mg) in N,N-dimethylformamide (3.5 ml), cyclohexyl 1-iodo-methylpropyl carbonate (172 mg) and cesium carbonate (172 mg) were added under cooling with ice and the mixture was stirred for an hour at room temperature. Water was added to the reaction mixture, followed by extracting with ethyl acetate three times, then washing with brine. After drying the organic layer with anhydrous sodium sulfate, the desiccant was filtered off and the solvents were removed in vacuo. The resulting residue was purified by silica gel column chromatography (eluent: chloroform/methanol=100:0 to 97:3) to give the titled compound (127 mg) as a brown oil.

MS (ESI/APCI Dual) m/z 625 [M+H]⁺

¹H NMR (300 MHz, CHLOROFORM-d) δ ppm 0.82 (d, J=6.7 Hz, 3H), 0.95 (dd, J=6.7, 3.0 Hz, 3H), 1.15-2.18 (m, 13H), 1.40, 1.41 (s, 9H), 2.61-3.10 (m, 10H), 3.81-3.92 (m, 1H), 4.06-4.20 (m, 1H), 4.40-4.67 (m, 1H), 5.18-5.38 (m, 1H), 6.44-6.51 (m, 1H), 7.12-7.20 (m, 1H), 7.27-7.34 (m, 2H), 7.35-7.43 (m, 1H), 7.94 (s, 1H)

(2) Synthesis of 1-{[(cyclohexyloxy)carbonyl]oxy}-2-methylpropyl (2S)-2-[(3S)-3-aminopyrrolidin-1-yl]-3-(4,5-dihydroimidazo[1,5-a]quinolin-3-yl)propanoate trihydrochloride

To a solution of 1-{[(cyclohexyloxy)carbonyl]oxy}-2-methylpropyl (2S)-2-{(3S)-3-[(tert-butoxycarbonyl)amino]pyrrolidin-1-yl}-(3-(4,5-dihydroimidazo[1,5-a]quinolin-3-yl)propanoate (127 mg) in ethyl acetate (1 ml), a 4 M HCl solution in ethyl acetate (1 ml) was added and the mixture was stirred for two hours at room temperature. The solvent was removed in vacuo and water was added to the resulting residue, which was subjected to azeotropic distillation to give the titled compound (compound 12; 110 mg) as a light brown amorphous mass.

MS (ESI/APCI Dual) m/z 525 [M+H]⁺

¹H NMR (600 MHz, DEUTERIUM OXIDE) δ ppm 0.80-0.89 (m, 6H), 1.03-1.31 (m, 5H), 1.37-1.75 (m, 5H), 1.96-2.11 (m, 2H), 2.42-2.59 (m, 1H), 2.83-3.14 (m, 5H), 3.20-3.51 (m, 4H), 3.99-4.09 (m, 1.5H), 4.13-4.19 (m, 0.5H), 4.22-4.30 (m, 0.5H), 4.45-4.53 (m, 0.5H), 6.39-6.44 (m, 1H), 7.46-7.54 (m, 3H), 7.72-7.75 (m, 1H), 9.37, 9.39 (s, 1H)

### Example 13

### Synthesis of (5-methyl-2-oxo-1,3-dioxol-4-yl)methyl (2S)-2-[(3S)-3-aminopyrrolidin-1-yl]-3-(4,5-dihydroimidazo[1,5-a]quinolin-3-yl)propanoate trihydrochloride

(1) Synthesis of (5-methyl-2-oxo-1,3-dioxol-4-yl)methyl (2S)-2-{(3S)-3-[(tert-butoxycarbonyl)amino]pyrrolidin-1-yl}-(3-(4,5-dihydroimidazo[1,5-a]quinolin-3-yl)propanoate

To a solution of (2S)-2-{(3S)-3-[(tert-butoxycarbonyl)amino]pyrrolidin-1-yl}-(3-(4,5-dihydroimidazo[1,5-a]quinolin-3-yl)propanoic acid (300 mg) in N,N-dimethylformamide (7 ml), cesium carbonate (342 mg) and 4-chloromethyl-5-methyl-1,3-dioxol-2-one (156 mg) were added under cooling with ice and the mixture was stirred for an hour at the same temperature and for an additional 6 hours at room temperature. Water was added to the reaction mixture under cooling with ice, followed by extracting with ethyl acetate. After washing the organic layer with water twice and with brine, drying was performed with anhydrous sodium sulfate; thereafter, the desiccant was filtered off and the solvents were removed in vacuo. The resulting residue was purified by silica gel column chromatography (eluent: chloroform/methanol=100:0 to 90:10) to give the titled compound (285 mg) as a light brown gum.

MS (ESI/APCI Dual) m/z 539 [M+H]⁺

¹H NMR (300 MHz, CHLOROFORM-d) δ ppm 1.42 (s, 9H), 1.58-1.72 (m, 1H), 2.09-2.24 (m, 1H), 2.13 (s, 3H), 2.55-2.68 (m, 1H), 2.69-2.78 (m, 1H), 2.80-3.07 (m, 8H), 3.73-3.81 (m, 1H), 4.07-4.21 (m, 1H), 4.73-4.88 (m, 2H), 5.11-5.21 (m, 1H), 7.13-7.20 (m, 1H), 7.25-7.34 (m, 2H), 7.39-7.44 (m, 1H), 7.93 (s, 1H)

(2) Synthesis of (5-methyl-2-oxo-1,3-dioxol-4-yl)methyl (2S)-2-[(3S)-3-aminopyrrolidin-1-yl]-3-(4,5-dihydroimidazo[1,5-a]quinolin-3-yl)propanoate trihydrochloride

To a solution of (5-methyl-2-oxo-1,3-dioxol-4-yl)methyl (2S)-2-{(3S)-3-[(tert-butoxycarbonyl)amino]pyrrolidin-1-yl}-(3-(4,5-dihydroimidazo[1,5-a]quinolin-3-yl)propanoate (285 mg) in ethyl acetate (5 ml), a 4 M HCl solution in ethyl acetate (5 ml) was added and the mixture was stirred for two hours at room temperature. The solvent was removed in vacuo and water was added to the resulting residue, which was subjected to azeotropic distillation to give the titled compound (compound 13; 247 mg) as a brown amorphous mass.

MS (ESI/APCI Dual) m/z 439 [M+H]⁺

¹H NMR (600 MHz, DEUTERIUM OXIDE) δ ppm 1.92-2.00 (m, 1H), 2.06 (s, 3H), 2.37-2.46 (m, 1H), 2.86-3.04 (m, 6H), 3.12-3.18 (m, 1H), 3.20-3.28 (m, 2H), 3.31-3.37 (m, 1H), 3.88-4.02 (m, 2H), 4.89 (d, J=14.2 Hz, 1 H), 5.05 (d, J=14.2 Hz, 1H), 7.43-7.52 (m, 3H), 7.69-7.74 (m, 1H), 9.28 (s, 1H)

### Example 14

### Synthesis of (5-tert-butyl-2-oxo-1,3-dioxol-4-yl)methyl (2S)-2-[(3S)-3-aminopyrrolidin-1-yl]-3-(4,5-dihydroimidazo[1,5-a]quinolin-3-yl)propanoate trihydrochloride

(1) Synthesis of (5-tert-butyl-2-oxo-1,3-dioxol-4-yl)methyl (2S)-2-{(3S)-3-[(tert-butoxycarbonyl)amino]pyrrolidin-1-yl}-(3-(4,5-dihydroimidazo[1,5-a]quinolin-3-yl)propanoate

A portion (132 mg) of the 4-(bromomethyl)-5-tert-butyl-1,3-dioxol-2-one synthesized in Reference Example 1 was dissolved in N,N-dimethylformamide (3 ml); to the resulting solution, (2S)-2-{(3S)-3-[(tert-butoxycarbonyl)amino]pyrrolidin-1-yl}-(3-(4,5-dihydroimidazo[1,5-a]quinolin-3-yl)propanoic acid (152 mg) and cesium carbonate (175 mg) were added under cooling with ice and the mixture was stirred for two hours at the same temperature. Ethyl acetate was added to the reaction mixture, followed by washing with brine twice. After drying the organic layer with anhydrous magnesium sulfate, the desiccant was filtered off and the solvents were removed in vacuo. The resulting residue was purified by silica gel column chromatography (eluent: ethyl acetate chloroform/methanol=9:1) to give the titled compound (99 mg) as a pale yellow gum.

MS (ESI/APCI Dual) m/z 581 [M+H]⁺

¹H NMR (600 MHz, CHLOROFORM-d) δ ppm 1.24 (s, 9H), 1.41 (s, 9H), 1.61-1.71 (m, 1H), 2.11-2.22 (m, 1H), 2.58-2.68 (m, 1H), 2.69-2.79 (m, 1H), 2.82-2.92 (m, 5H), 2.93-3.06 (m, 3H), 3.79-3.84 (m, 1H), 4.12-4.19 (m, 1H), 4.90 (s, 2H), 5.14-5.21 (m, 1H), 7.15-7.18 (m, 1 H), 7.27-7.33 (m, 2H), 7.40-7.43 (m, 1H), 7.94 (s, 1 H)

(2) Synthesis of (5-tert-butyl-2-oxo-1,3-dioxol-4-yl)methyl (2S)-2-[(3S)-3-aminopyrrolidin-1-yl]-3-(4,5-dihydroimidazo[1,5-a]quinolin-3-yl)propanoate trihydrochloride

An aqueous solution of 4 M HCl (3 ml) having (5-tert-butyl-2-oxo-1,3-dioxol-4-yl)methyl (2S)-2-{(3S)-3-[(tert-butoxycarbonyl)amino]pyrrolidin-1-yl}-(3-(4,5-dihydroimidazo[1,5-a]quinolin-3-yl)propanoate (96 mg) dissolved therein was stirred for 1.5 hours at room temperature. The solvent was removed in vacuo to give the titled compound (compound 14; 90 mg) as a light brown amorphous mass.

MS (ESI/APCI Dual) m/z 481 [M+H]⁺

¹H NMR (600 MHz, DEUTERIUM OXIDE) δ ppm 1.14 (s, 9H), 2.06-2.14 (m, 1H), 2.50-2.59 (m, 1H), 2.88-2.95 (m, 1H), 2.96-3.07 (m, 3H), 3.17-3.23 (m, 1H), 3.26-3.31 (m, 1H), 3.34-3.40 (m, 1H), 3.41-3.51 (m, 2H), 3.53-3.58 (m, 1H), 4.06-4.13 (m, 1H), 4.21-4.26 (m, 1H), 5.03 (d, J=14.2 Hz, 1H), 5.18 (d, J=14.2 Hz, 1H), 7.44-7.52 (m, 3H), 7.69-7.73 (m, 1H), 9.37 (s, 1H)

Optical purity: >99% ee

r.t.: 34.37 min

### Example 15

### Synthesis of [5-(2-methylpropyl)-2-oxo-1,3-dioxol-4-yl]methyl (2S)-2-[(3S)-3-aminopyrrolidin-1-yl]-3-(4,5-dihydroimidazo[1,5-a]quinolin-3-yl)propanoate trihydrochloride

(1) Synthesis of [5-(2-methylpropyl)-2-oxo-1,3-dioxol-4-yl]methyl (2S)-2-{(3S)-3-[(tert-butoxycarbonyl)amino]pyrrolidin-1-yl}-(3-(4,5-dihydroimidazo[1,5-a]quinolin-3-yl)propanoate

A portion (127 mg) of the 4-(bromomethyl)-5-(2-methylpropyl)-1,3-dioxol-2-one synthesized in Reference Example 2 was dissolved in N,N-dimethylformamide (2 ml); to the resulting solution, (2S)-2-{(3S)-3-[(tert-butoxycarbonyl)amino]pyrrolidin-1-yl}-(3-(4,5-dihydroimidazo[1,5-a]quinolin-3-yl)propanoic acid (150 mg) and cesium carbonate (176 mg) were added under cooling with ice and the mixture was stirred for three hours at the same temperature. Ethyl acetate was added to the reaction mixture, followed by washing with brine twice. After drying the organic layer with anhydrous magnesium sulfate, the desiccant was filtered off and the solvents were removed in vacuo. The resulting residue was purified by silica gel column chromatography (eluent: ethyl acetate) to give the titled compound (139 mg) as a pale yellow gum.

MS (ESI/APCI Dual) m/z 581 [M+H]⁺

¹H NMR (300 MHz, CHLOROFORM-d) δ ppm 0.92 (d, J=6.7 Hz, 6H), 1.42 (s, 9H), 1.59-1.74 (m, 1H), 1.83-1.99 (m, 1H), 2.07-2.25 (m, 1H), 2.31 (d, J=7.1 Hz, 2H), 2.54-2.68 (m, 1H), 2.69-2.79 (m, 1H), 2.80-3.06 (m, 8H), 3.74-3.84 (m, 1H), 4.07-4.22 (m, 1H), 4.80 (s, 2H), 5.10-5.24 (m, 1H), 7.13-7.21 (m, 1H), 7.27-7.34 (m, 2H), 7.39-7.45 (m, 1H), 7.93 (s, 1H)

(2) Synthesis of [5-(2-methylpropyl)-2-oxo-1,3-dioxol-4-yl]methyl (2S)-2-[(3S)-3-aminopyrrolidin-1-yl]-3-(4,5-dihydroimidazo[1,5-a]quinolin-3-yl)propanoate trihydrochloride

An aqueous solution of 4 M HCl (1.5 ml) having [5-(2-methylpropyl)-2-oxo-1,3-dioxol-4-yl]methyl (2S)-2-{(3S)-3-[(tert-butoxycarbonyl)amino]pyrrolidin-1-yl}-(3-(4,5-dihydroimidazo[1,5-a]quinolin-3-yl)propanoate (137 mg) dissolved therein was stirred for an hour at room temperature. The solvent was removed in vacuo to give the titled compound (compound 15; 131 mg) as a light brown amorphous mass.

MS (ESI/APCI Dual) m/z 481 [M+H]⁺

¹H NMR (600 MHz, DEUTERIUM OXIDE) δ ppm 0.79 (d, J=6.9 Hz, 3H), 0.80 (d, J=6.9 Hz, 3H), 1.74-1.83 (m, 1H), 2.02-2.13 (m, 1H), 2.24-2.31 (m, 2H), 2.47-2.56 (m, 1H), 2.88-2.97 (m, 1H), 2.97-3.07 (m, 3H), 3.09-3.18 (m, 1H), 3.19-3.27 (m, 1H), 3.29-3.53 (m, 4H), 4.03-4.10 (m, 1H), 4.12-4.21 (m, 1H), 4.95 (d, J=14.2 Hz, 1H), 5.10 (d, J=14.2 Hz, 1H), 7.43-7.53 (m, 3H), 7.69-7.73 (m, 1H), 9.34 (s, 1H)

### Example 16

### Synthesis of (5-cyclohexyl-2-oxo-1,3-dioxol-4-yl)methyl (2S)-2-[(3S)-3-aminopyrrolidin-1-yl]-3-(4,5-dihydroimidazo[1,5-a]quinolin-3-yl)propanoate trihydrochloride

(1) Synthesis of (5-cyclohexyl-2-oxo-1,3-dioxol-4-yl)methyl (2S)-2-{(3S)-3-[(tert-butoxycarbonyl)amino]pyrrolidin-1-yl}-(3-(4,5-dihydroimidazo[1,5-a]quinolin-3-yl)propanoate

A portion (106 mg) of the 4-(bromomethyl)-5-cyclohexyl-1,3-dioxol-2-one synthesized in Reference Example 3 was dissolved in N,N-dimethylformamide (2 ml); to the resulting solution, (2S)-2-[(3S)-3-[(tert-butoxycarbonyl)amino]pyrrolidin-1-yl]-(3-(4,5-dihydroimidazo[1,5-a]quinolin-3-yl)propanoic acid (155 mg) and cesium carbonate (132 mg) were added under cooling with ice and the mixture was stirred for five hours at the same temperature. Ethyl acetate was added to the reaction mixture, followed by washing with brine twice. After drying the organic layer with anhydrous magnesium sulfate, the desiccant was filtered off and the solvents were removed in vacuo. The resulting residue was purified by silica gel column chromatography (eluent: chloroform/methanol=100:0 to 97:3) to give the titled compound (144 mg) as a pale yellow gum.

MS (ESI/APCI Dual) m/z 607 [M+H]⁺

¹H NMR (600 MHz, CHLOROFORM-d) δ ppm 1.15-1.23 (m, 1H), 1.25-1.34 (m, 2H), 1.42 (s, 9H), 1.42-1.48 (m, 1H), 1.62-1.83 (m, 7H), 2.10-2.20 (m, 1H), 2.51-2.57 (m, 1H), 2.58-2.66 (m, 1H), 2.71-2.77 (m, 1H), 2.82-3.05 (m, 8H), 3.76-3.82 (m, 1H), 4.12-4.19 (m, 1H), 4.83 (s, 2H), 5.12-5.20 (m, 1H), 7.14-7.19 (m, 1H), 7.27-7.32 (m, 2H), 7.40-7.43 (m, 1H), 7.93 (s, 1H)

(2) Synthesis of (5-cyclohexyl-2-oxo-1,3-dioxol-4-yl)methyl (2S)-2-[(3S)-3-aminopyrrolidin-1-yl]-3-(4,5-dihydroimidazo[1,5-a]quinolin-3-yl)propanoate trihydrochloride

An aqueous solution of 4 M HCl (3 ml) having (5-cyclohexyl-2-oxo-1,3-dioxol-4-yl)methyl (2S)-2-{(3S)-3-[(tert-butoxycarbonyl)amino]pyrrolidin-1-yl}-(3-(4,5-dihydroimidazo[1,5-a]quinolin-3-yl)propanoate (141 mg) dissolved therein was stirred for an hour at room temperature. The solvent was removed in vacuo to give the titled compound (compound 16; 129 mg) as a light brown amorphous mass.

MS (ESI/APCI Dual) m/z 507 [M+H]⁺

¹H NMR (600 MHz, DEUTERIUM OXIDE) δ ppm 1.05-1.15 (m, 1H), 1.15-1.33 (m, 4H), 1.54-1.64 (m, 3H), 1.64-1.70 (m, 2H), 2.08-2.16 (m, 1H), 2.51-2.60 (m, 2H), 2.87-2.94 (m, 1H), 2.98-3.07 (m, 3H), 3.20-3.27 (m, 1H), 3.29-3.40 (m, 2H), 3.45-3.53 (m, 2H), 3.56-3.62 (m, 1H), 4.08-4.15 (m, 1H), 4.25-4.31 (m, 1H), 4.96 (d, J=14.2 Hz, 1H), 5.14 (d, J=14.2 Hz, 1H), 7.44-7.53 (m, 3H), 7.69-7.73 (m, 1H), 9.35 (s, 1H)

### Example 17

### Synthesis of (5-benzyl-2-oxo-1,3-dioxol-4-yl)methyl (2S)-2-[(3S)-3-aminopyrrolidin-1-yl]-3-(4,5-dihydroimidazo[1,5-a]quinolin-3-yl)propanoate trihydrochloride

(1) Synthesis of (5-benzyl-2-oxo-1,3-dioxol-4-yl)methyl (2S)-2-{(3S)-3-[(tert-butoxycarbonyl)amino]pyrrolidin-1-yl}-(3-(4,5-dihydroimidazo[1,5-a]quinolin-3-yl)propanoate

A portion (92 mg) of the 4-benzyl-5-(bromomethyl)-1,3-dioxol-2-one synthesized in Reference Example 4 was dissolved in N,N-dimethylformamide (2 ml); to the resulting solution, (2S)-2-{(3S)-3-[(tert-butoxycarbonyl)amino]pyrrolidin-1-yl}-(3-(4,5-dihydroimidazo[1,5-a]quinolin-3-yl)propanoic acid (146 mg) and cesium carbonate (110 mg) were added under cooling with ice and the mixture was stirred for three hours at the same temperature. Ethyl acetate was added to the reaction mixture, followed by washing with brine twice. After drying the organic layer with anhydrous magnesium sulfate, the desiccant was filtered off and the solvents were removed in vacuo. The resulting residue was purified by silica gel column chromatography (eluent: ethyl acetate→chloroform/methanol=97:3) to give the titled compound (125 mg) as a pale yellow gum.

MS (ESI/APCI Dual) m/z 615 [M+H]⁺

¹H NMR (600 MHz, CHLOROFORM-d) δ ppm 1.42 (s, 9H), 1.59-1.69 (m, 1H), 2.10-2.21 (m, 1H), 2.57-2.65 (m, 1H), 2.69-2.76 (m, 1H), 2.77-3.05 (m, 8H), 3.74-3.82 (m, 3H), 4.10-4.18 (m, 1H), 4.78 (s, 2H), 5.10-5.20 (m, 1H), 7.14-7.18 (m, 1H), 7.19-7.22 (m, 2H), 7.22-7.26 (m, 1H), 7.27-7.32 (m, 4H), 7.37-7.40 (m, 1H), 7.90 (s, 1H)

(2) Synthesis of (5-benzyl-2-oxo-1,3-dioxol-4-yl)methyl (2S)-2-[(3S)-3-aminopyrrolidin-1-yl]-3-(4,5-dihydroimidazo[1,5-a]quinolin-3-yl)propanoate trihydrochloride

An aqueous solution of 4 M HCl (3 ml) having (5-benzyl-2-oxo-1,3-dioxol-4-yl)methyl (2S)-2-{(3S)-3-[(tert-butoxycarbonyl)amino]pyrrolidin-1-yl}-(3-(4,5-dihydroimidazo[1,5-a]quinolin-3-yl)propanoate (124 mg) dissolved therein was stirred for an hour at room temperature. The solvent was removed in vacuo to give the titled compound (compound 17; 114 mg) as a light brown amorphous mass.

MS (ESI/APCI Dual) m/z 515 [M+H]⁺

¹H NMR (600 MHz, DEUTERIUM OXIDE) δ ppm 2.04-2.12 (m, 1H), 2.49-2.56 (m, 1H), 2.67-2.79 (m, 2H), 2.80-2.91 (m, 2H), 3.10-3.17 (m, 1H), 3.22-3.26 (m, 1H), 3.28-3.34 (m, 1H), 3.36-3.45 (m, 2H), 3.47-3.53 (m, 1H), 3.74-3.83 (m, 2H), 4.05-4.10 (m, 1H), 4.14-4.18 (m, 1H), 4.93 (d, J=14.2 Hz, 1H), 5.11 (d, J=14.2 Hz, 1H), 7.22-7.25 (m, 2H), 7.28-7.31 (m, 1H), 7.33-7.37 (m, 2H), 7.40-7.43 (m, 1H), 7.43-7.47 (m, 2H), 7.48-7.52 (m, 1H), 9.10 (s, 1H)

### Example 18

### Synthesis of (5-tert-butyl-2-oxo-1,3-dioxol-4-yl)methyl (2S)-3-(4,5-dihydroimidazo[1,5-a]quinolin-3-yl)-2-{(3S)-3-[({1-[(2-methylpropanoyl)oxy]ethoxy}carbonyl)amino]pyrrolidin-1-yl}propanoate

A portion (153 mg) of the (5-tert-butyl-2-oxo-1,3-dioxol-4-yl)methyl (2S)-2-[(3S)-3-aminopyrrolidin-1-yl]-3-(4,5-dihydroimidazo[1,5-a]quinolin-3-yl)propanoate trihydrochloride synthesized in Example 14 was dissolved in N,N-dimethylformamide (3 ml); to the resulting solution, 1-{[4-nitrophenoxy)carbonyl]oxy}ethyl 2-methyl propanoate (90 mg) and triethylamine (110 μl) were added dropwise under cooling with ice and the mixture was stirred for 11 hours at room temperature. Ethyl acetate was added to the reaction mixture, followed by washing with brine twice. After drying the organic layer with anhydrous magnesium sulfate, the desiccant was filtered off and the solvents were removed in vacuo. The resulting residue was purified by silica gel column chromatography (eluent: chloroform/methanol=99:1 to 95:5) to give the titled compound (compound 18; 112 mg) as a colorless solid.

MS (ESI/APCI Dual) m/z 639 [M+H]⁺

¹H NMR (600 MHz, CHLOROFORM-d) S ppm 1.12-1.18 (m, 6H), 1.25 (s, 9H), 1.38-1.52 (m, 3H), 1.65-1.76 (m, 1H), 2.08-2.23 (m, 1H), 2.45-2.58 (m, 1H), 2.59-2.70 (m, 1H), 2.79-3.06 (m, 10H), 3.78-3.86 (m, 1H), 4.13-4.21 (m, 1H), 4.91 (s, 2H), 5.63-5.77 (m, 1H), 6.74-6.83 (m, 1H), 7.13-7.20 (m, 1H), 7.27-7.35 (m, 2H), 7.39-7.45 (m, 1H), 7.94 (s, 1H)

The structural formulas of compounds 1-4 prepared in the Reference Examples are shown in the following Table 1-1. The structural formulas of compounds 1-18 in the Examples of the invention are shown in the following Table 1-2.

TABLE 1-1

| Ref. No. | Structure |
|---|---|
| 1 | 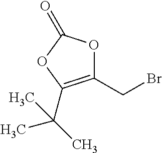 |
| 2 | 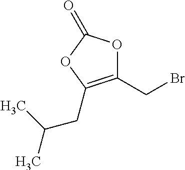 |
| 3 | 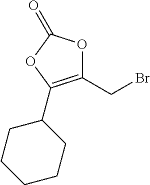 |
| 4 | 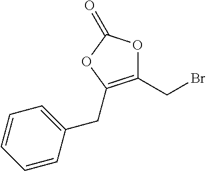 |

TABLE 1-2

| Ex. | | |
|---|---|---|
| No | Structure | Salt |
| 1 | 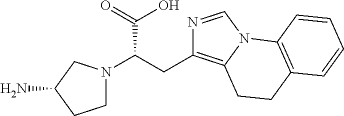 | 3HCl |
| 2 | 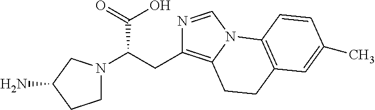 | 3HCl |
| 3 | 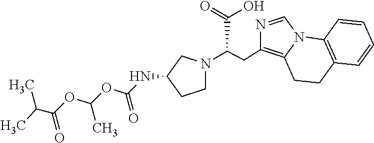 | — |
| 4 | 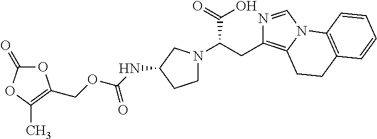 | — |
| 5 | 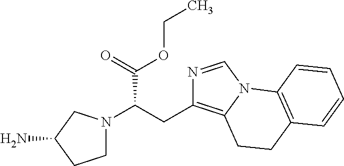 | 3HCl |
| 6 | 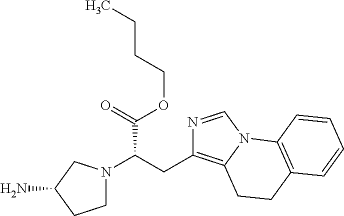 | 3HCl |
| 7 | 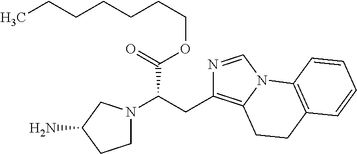 | 3HCl |
| 8 | 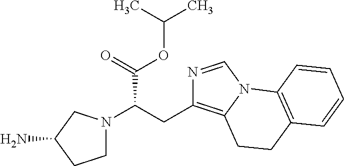 | 3HCl |
| 9 | 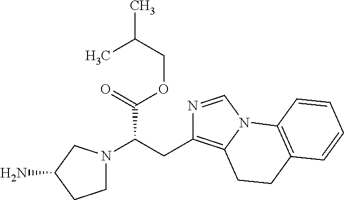 | 3HCl |
| 10 | 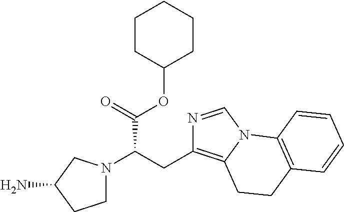 | 3HCl |
| 11 | 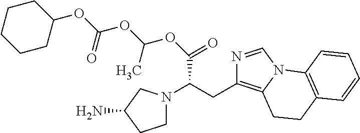 | 3HCl |
| 12 | 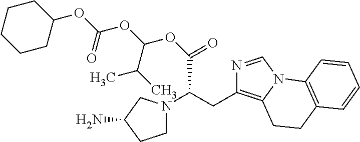 | 3HCl |
| 13 | 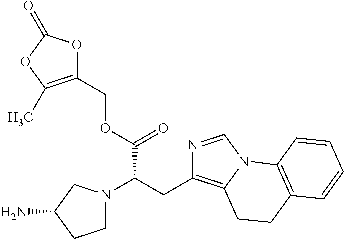 | 3HCl |
| 14 | 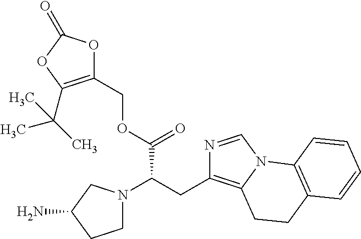 | 3HCl |
| 15 | 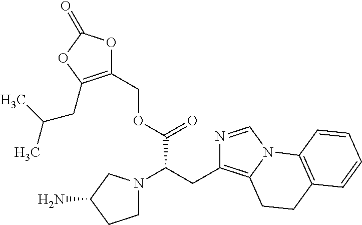 | 3HCl |
| 16 | 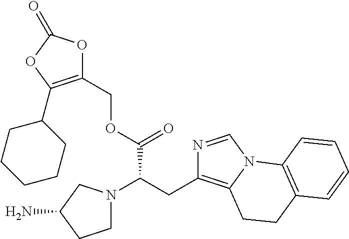 | 3HCl |
| 17 | 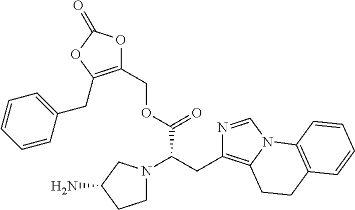 | 3HCl |
| 18 | 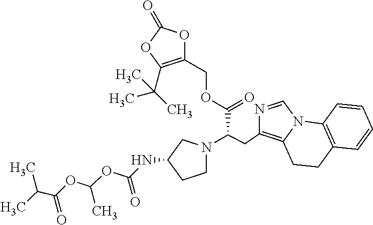 | — |

Test 1 [TAFIa Inhibition Test]

Compounds of the present invention were measured for their TAFIa inhibitory activity as follows based on the method described in Thromb. Haemost. 79, 371-377 (1998).

### (a) Preparation of TAFIa Solution

To 450 μl of TAFI (the product of Enzyme Research Laboratories whose concentration was adjusted to 18 μg/ml with buffer A: 100 mM Tris-HCl, pH 7.4), 45 μl of a thrombomodulin solution (a rabbit lung derived thrombomodulin produced by American Diagnostica whose concentration was adjusted to 1 μg/ml with buffer B: 50 mM Tris-HCl, pH 7.5, containing 0.15 M NaCl) and 45 μl of a thrombin solution (a freeze-dried human plasma derived thrombin produced by Sigma and dissolved in water to have a concentration of 30 μU/ml) were added and the mixture was left to stand at room temperature for 25 minutes.

### (b) Method of Measuring TAFIa Inhibitory Activity

To wells on a 96-well microplate, the above-prepared TAFIa solution, a test compound, and a substrate solution (Hip-Arg produced by Sigma and dissolved in buffer C of 100 mM Tris-HCl, pH 8.3, to have a concentration of 3.6 mM) were added in respective amounts of 20 μl/well, 10 μl/well, and 70 μl/well. The individual components were mixed well and the reaction was carried out for 40 minutes at room temperature.

Subsequently, a color former (1% cyanuric chloride in 1,4-dioxane) was added in an amount of 50 μl to each well and the plate was left to stand for 3 minutes at room temperature, then absorbance at 405 nm was measured with a microplate reader (Spectramax M2 of Molecular Devices). With the absorbance in the absence of a test compound minus the absorbance in the absence of an enzyme being taken as 100%, the concentration of the compound that inhibited 50% of the reaction (IC₅₀) was calculated from the absorbance in the presence of the test compound minus the absorbance in the absence of the enzyme.

For two compounds of the present invention, the above test was conducted and on the basis of the results of measurements, TAFIa inhibitory activity was calculated to give the results shown in Table 2.

TABLE 2

| Ex. No. | IC₅₀ (nM) |
|---|---|
| 1 | 36 |
| 2 | 23 |

### Test 2 [Measurement of In Vivo Exposure Based on Plasma Level in Rats]

To determine the in vivo exposure, compound 14 as an exemplary prodrug compound of the present invention and compound 1 as its parent compound were orally administered to rats and the plasma level of compound 1 was measured as described below for comparative purposes.

Seven-week old rats (220-280 g; male; lineage; Crl:CD (SD)) purchased from Charles River Laboratories Japan Inc. were acclimatized for at least two days before they were administered with compounds of the present invention. Compound 14 was dissolved in a solvent of administration at a concentration equivalent to 2 mg/mL as calculated for the parent compound 1 and it was then administered orally in an amount equivalent to 10 mg/kg of that parent compound. Half an hour and four hours later, blood was taken from the tail vein of each rat through a blood collecting tube (EDTA treated (for compound 1) or both EDTA treatment and dichlorvos addition (for compound 14)) and immediately centrifuged (12,000×g at 4° C. for 2 minutes (compound 1) or 3 minutes (compound 14)), to recover plasma samples, which were stored frozen at −30° C. After thawing the plasma samples under cooling on ice, a solution of each internal standard substance was added, followed by deproteinization and centrifugation (3639×g at 4° C. for 10 minutes). The concentration of the parent compound 1 in the supernatant was measured by LC/MS/MS.

As the following Table 3 summarizes, the administration of the prodrug compound of the present invention showed higher plasma levels of the parent compound, indicating higher in vivo exposures of the parent compound. Therefore, by administering the prodrug compound of the present invention, the physiological action of the parent compound will be exhibited more effectively than the parent compound.

TABLE 3
Comparison Between Plasma Levels of
Compound 1 (Parent Compound) and Compound
14 According to the Present Invention

| | Plasma Level of Compound | |
|---|---|---|
| | 1 (Parent Compound) | |
| Compound | (ng/mL) | |
| (10 mg/kg p.o.) | 0.5 hr later | 4 hrs later |
| Compound 1 | 93 | 47 |
| (parent compound) | | |
| Compound 14 | 1200 | 584 |

Administration solvent for compound 1: physiological saline
Administration solvent for compound 14: 0.01M HCL in water
Internal Standard Substance for compound 1: compound A (MeCN/MeOH (9/1))
Internal Standard Substance for compound 14: compound B (10% TCA)

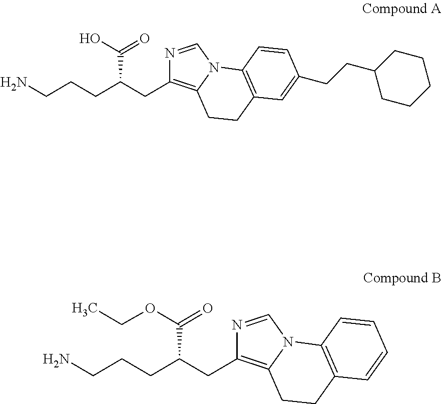

Compounds A and B used as the internal standard substances were synthesized by the methods described in PCT/JP2009/068526 (see Examples 24 and 32, respectively).

### INDUSTRIAL APPLICABILITY

The present invention provides pharmaceuticals that have sufficiently high TAFIa inhibitory activity to be effective for preventing or treating thrombus-derived diseases and the like, and it is therefore expected to relieve the burden on patients and contribute to the progress of the pharmaceutical industry.

1. A dihydroimidazoquinoline compound represented by the following formula (I), or a pharmaceutically acceptable salt thereof: 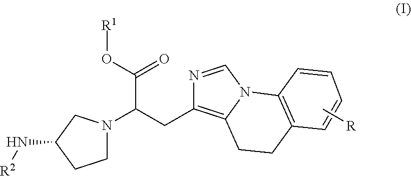
wherein R is a hydrogen atom or a C₁₋₁₀ alkyl group;
R¹ is a hydrogen atom, a C₁₋₁₀ alkyl group, a C₃₋₈ cycloalkyl group or a substituent having the structure represented by the following formula Ia or Ib: 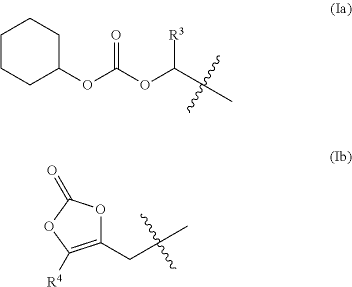
where R³ is a C₁₋₆ alkyl group;
R⁴ is a C₁₋₆ alkyl group, a C₃₋₈ cycloalkyl group, or a benzyl group; and
R² is a hydrogen atom or a substituent having the structure represented by the following formula Ic or Id:

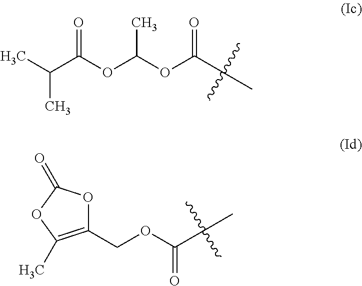

2. The compound of claim 1, which is a dihydroimidazoquinoline compound represented by the following formula (II), or a pharmaceutically acceptable salt thereof: 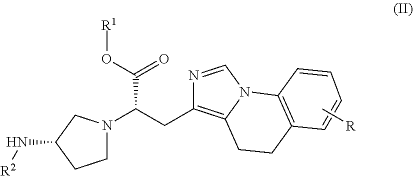
wherein R, R¹ and R² are as defined in claim 1.

3. The compound of claim 2, which is a dihydroimidazoquinoline compound represented by the following formula (III), or a pharmaceutically acceptable salt thereof: 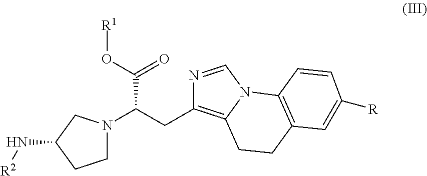
wherein R, R¹ and R² are as defined in claim 2.

4. The compound of claim 3, which is a dihydroimidazoquinoline compound represented by the following formula (IV), or a pharmaceutically acceptable salt thereof: 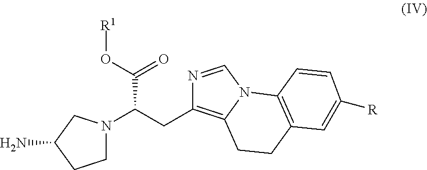
wherein R and R¹ are as defined claim 3.

5. The compound of claim 3, which is a dihydroimidazoquinoline compound represented by the following formula (V), or a pharmaceutically acceptable salt thereof: 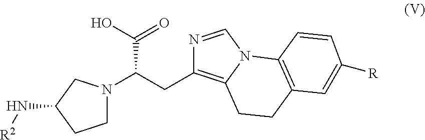
wherein R and R² are as defined in claim 3.

6. The compound of claim 1, which is a compound represented by the following formula (VI), or a pharmaceutically acceptable salt thereof: 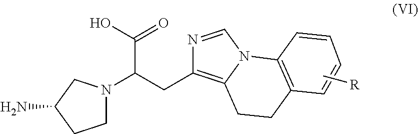
wherein R is as defined in claim 1.

7. The compound of claim 6, which is a dihydroimidazoquinoline compound represented by the following formula (VII), or a pharmaceutically acceptable salt thereof: 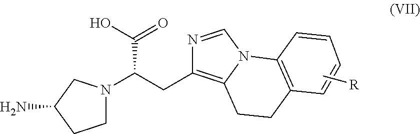
wherein R is as defined in claim 6.

8. The compound of claim 7, which is a dihydroimidazoquinoline compound represented by the following formula (VIII), or a pharmaceutically acceptable salt thereof: 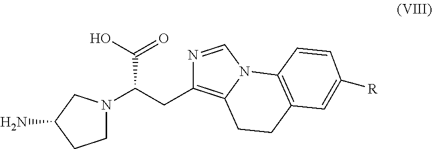
wherein R is as defined in claim 7.

9. A TAFIa inhibitor comprising the compound or the pharmaceutically acceptable salt thereof defined in claim 1, as an active ingredient.

10. An agent for preventing or treating a clot-derived disease, that comprises the compound or the pharmaceutically acceptable salt thereof defined in claim 1, as an active ingredient.
